(19) United States
(12) Patent Application Publication
(10) Pub. No.: US 2004/0009509 A1
(43) Pub. Date: Jan. 15, 2004

(54) Isolated human proteins that show high homology to human disease proteins, nucleic acid molecules encoding these human proteins, and uses thereof

(75) Inventor: Gangadharan Subramanian, Columbia, MD (US)

Correspondence Address: CELERA GENOMICS CORP., ATTN: WAYNE MONTGOMERY, VICE PRES, INTEL PROPERTY, 45 WEST GUDE DRIVE, C2-4#20, ROCKVILLE, MD, 20850, US

(73) Assignee: APPLERA CORPORATION, Norwalk, CT

(21) Appl. No.: 10/420,940

(22) Filed: Apr. 23, 2003

Related U.S. Application Data
(60) Provisional application No. 60/374,494, filed on Apr. 23, 2002.

(51) Int. Cl. C12Q 1/68; C07H 21/04; C12N 9/16; C12P 21/02; C12N 5/06

(52) U.S. Cl. 435/6; 435/69.1; 435/196; 435/320.1; 536/23.2; 435/325

(57) ABSTRACT

The present invention provides amino acid sequences of proteins that are encoded by genes within the human genome that show high homology to known human disease proteins, the PDG encoded proteins of the present invention. The present invention specifically provides isolated protein and nucleic acid molecules, and uses of these compositions, such as methods and kits to identify modulators of the PDG encoded proteins.

### FIELD OF THE INVENTION

The present invention is in the field of paralogous disease gene (“PDG”) encoded proteins, recombinant DNA molecules and protein production. The present invention specifically provides novel PDG encoded proteins and nucleic acid molecules encoding such protein molecules that show high homology to known human disease proteins, for use in the development of human diagnostics and therapeutic products.

### BACKGROUND OF THE INVENTION

The human genome comprises 24 different chromosomes, including 22 autosomes plus the X and Y sex chromosomes. The human genome comprises genes that encode all of the proteins in the human body and control all aspects of human life, such as embryogenesis, development, growth, reproduction, behavior, aging, metabolism, and many other processes. Furthermore, the human genome contains many regulatory and control elements and mechanisms, the vast majority of which are poorly understood or have not been identified. Regulatory and control elements (also referred to as expression modulating fragments (“EMFs”)) include, but are not limited to, promoters, enhancers, silencers, and insulators (see Bell et al., Science, Jan. 19, 2001, 291: 447-450 for a review of insulators).

A major effort of the worldwide biomedical community is currently directed at annotating the human genome and making sense of the primary sequence information, including gene discovery and identification of regulatory and control elements, as well as identification of other important features of non-coding DNA. Such annotation can be accomplished using a variety of presently available computer software and algorithms. For example, the Genscan and GeneWise programs are commonly used in the art for predicting genes and exon-intron structures in genomic sequences. However, the lack of an assembled reference sequence representing the majority of the human genome in a useful, easily accessible form has slowed such annotation efforts. For example, gene annotation computer programs typically are not adept at handling unfinished sequence that may have errors or gaps; such gaps/errors may disrupt the reading frame and cause frameshift errors. Therefore, the availability of high quality sequence representing the majority of the human genome would significantly improve the identification and characterization of commercially important genes.

Furthermore, a substantial proportion of current gene discovery efforts is directed at mining EST databases, on the assumption that any transcript represents a gene. However, the extent of transcripts that have no meaningful function, and may simply be the by-products of spurious transcription, has not been determined. Consequently, it has been estimated that EST databases may contain as little as 40% of the protein-coding portion of the human genome (Aparicio, Nature Genetics, June 2000, 25: 129-130). As a result, the primary sequence of the human genome represents the best source of identifying such protein-coding sequences that are not represented in EST databases. Therefore, the primary sequence of the human genome, in a useful, easily accessible form amenable to computer analysis, is needed for efforts directed at identifying the majority of protein-coding sequences throughout the human genome, as well as for accomplishing other annotation objectives.

Additionally, many expressed sequences, such as those in EST databases and other gene expression databases (such as databases of gene expression in various normal or diseased tissues), are not mapped. The availability of an assembled human genome sequence would enable expressed sequences to be mapped to the human genome by computerized alignment of expressed mRNA sequences with the human genome sequence; such mapping would provide the sequence surrounding the expressed gene, including 5′ and 3′ UTR regions. The sequence surrounding the gene could then be analyzed for control/regulatory elements or EMFs (such as promoters, enhancers, silencers, and insulators) that are involved in regulating the expression of the gene. Such elements may be identified, for example, based on sequence similarities to known EMF sequence motifs. Furthermore, this would enable the identification of nearby SNPs that may affect the expression of a particular gene or SNPs that may be associated with a particular disease. Additionally, multiple mRNA sequences that map to the same location in the human genome would readily be identified as potential alternative splice forms of the same gene.

Current gene annotation efforts are further limited in that it is likely that many human genes are much longer than previously anticipated. Consequently, the algorithms underlying exon-prediction computer programs used to identify genes have generally been based on shorter gene lengths than what likely exists in the human genome. As a result, large areas of the human genome thought to be intergenic “junk” DNA regions may instead be intragenic introns of extremely large genes (Wong et al, Genome Research, November 2000, 10: 1672-1678). Such large genes are being overlooked by current gene discovery efforts and are difficult to identify from genome data lacking long-range contiguity. Therefore, the availability of the assembled human genome in a useful, easily accessible form would greatly improve discovery and annotation of large genes, many of which are likely to be commercially valuable, and vastly enhance our understanding of the organization of the human genome. Furthermore, annotation of vast stretches of previously unannotated human genome sequence could be accomplished.

Increasingly, comparisons between genomes of different species is being utilized to discover regions of the human genome that are highly conserved between humans and other species, including both distantly related species, such as Drosophila, and more closely related species, such as the mouse or various primates. Such inter-species comparisons will greatly enhance the efficiency and accuracy of human genome annotation and identification of commercially valuable genes and genetic elements. However, such efforts are limited by the lack of an assembled human genome reference sequence that is in a useful form for efficient comparative genomic analysis.

The advantages of comparative genomic studies between the human genome and the genomes of other species can be illustrated using Drosophila as an example. Model organisms, such as Drosophila melanogaster, share many genes with humans whose sequences and functions have been conserved. In addition to myriad similarities in cellular structure and function, humans and Drosophila share pathways for intercellular signaling, developmental patterning, learning and behavior, as well as tumor formation and metastasis. Drosophila studies have provided the widest knowledge base available for any single organism; accordingly, Drosophila is widely used by developmental biologists to identify and characterize the activity of genes with similar functions in humans.

The genes involved in the development of Drosophila, with few exceptions, are the same as those involved in the development of higher organisms. Developmental biology studies the sequential activation and interaction of genes, in relation to developing morphology. For Drosophila, one can begin with a list of genes active in the egg and follow the morphological changes and gene activation through to adulthood. If one knows the genes involved in the development of Drosophila, one also knows, to a reasonable approximation, the genes involved in the development of humans. A human genome discovery system that provides the assembled human genome in an easily accessible form would be useful for comparative genomic studies, such as between human and Drosophila genomes, thereby significantly enhancing the identification of commercially valuable human genes and therapeutic/pharmaceutical agents.

A major goal in therapeutic/pharmaceutical drug development is to understand and elucidate the molecular mechanisms that govern such processes as cell signaling, cell-cell interactions, gene interactions, and disease pathways. Many of these molecular mechanisms can be elucidated through studies of the human genome sequence, specifically in the comparison and contrast of the roles of paralogous disease genes. Consequently, therapeutic/pharmaceutical drug development would greatly benefit from a human genome discovery system.

Another aspect of therapeutic/pharmaceutical drug development, and disease diagnosis and treatment, involves correlating sequence variations in the human genome, such as single nucleotide polymorphisms (“SNPs”), with individual variation in disease susceptibility, severity and progression, and individual variation in response to particular treatments or drugs (i.e., “pharmacogenomics”). Pharmacogenomics is currently a major area of research and development within the biotechnology industry and holds a great deal of promise for disease treatment and diagnosis. However, only a limited proportion of all human sequence variants have been discovered to date, due to the large amount of work required for their detection by conventional methods. Discovery of novel sequence variations in the human genome would be greatly enhanced by a human genome discovery system that could provide a reference human genome sequence in a format for efficient computer analysis. Such a discovery system would be useful for comparisons between sequences from multiple individuals, such as individuals of diverse ethnicity or individuals having a particular phenotype or disease of interest, thereby enabling the discovery of novel sequence variants. Furthermore, such a discovery system would provide mapping information for such sequence variants. Additionally, the sequence variations between paralogous genes can illustrate the roles of these genes in disease diagnosis and treatment.

Oligonucleotide probes have long been used to detect complementary nucleic acid sequences in a nucleic acid of interest (the “target” nucleic acid). In some assay formats, the oligonucleotide probe is tethered, i.e., by covalent attachment, to a solid support, and arrays of oligonucleotide probes immobilized on solid supports have been used to detect specific nucleic acid sequences in a target nucleic acid. See, e.g., PCT patent publication Nos. WO 89/10977 and 89/11548. The development of arraying technologies such as photolithographic synthesis of a nucleic acid array and high density spotting of cDNA products has provided methods for making very large arrays of oligonucleotide probes in very small areas. See U.S. Pat. No. 5,143,854 and PCT patent publication Nos. WO 90/15070 and 92/10092. Microfabricated arrays of large numbers of oligonucleotide probes, (often referred to as “DNA chips” or “biochips”), offer great promise for a wide variety of applications. Such arrays may contain, for example, thousands or millions of oligonucleotide probes.

However, such high-density arrays require a means to efficiently design the large numbers of array probes that need to be synthesized for analysis of the human genome, or selected portions thereof. A human genome discovery system that provides the human genome sequence in a useable, efficiently accessible form, such as in a computer-based system, would greatly facilitate and automate the design of large numbers of probes capable of covering the complete human genome, or selected portions thereof, for analysis. A set of PDGs can further facilitate efficient probe design, enabling an array to distinguish a PDG from its associated known disease gene. These probes can then be, for example, synthesized and immobilized on a solid support for array-based genome analysis.

Thus, a definite need exists for a discovery system that provides the sequence of the human genome in a form that provides an efficient tool for understanding, characterizing, and annotating the complete human genome. The primary sequence of the human genome in a useable form would be invaluable for identifying human therapeutic targets and commercially valuable genes and genetic elements, such as regulatory/control elements, and for developing human therapeutic agents. Not only will the system serve as a basis for gene discovery and validation, the system will aid in the understanding of complex genetic mechanisms that control such cellular functions as cell differentiation, proliferation, and death. Furthermore, such a human genome discovery system would provide a reference for characterizing all genes, as well as other features, such as regulatory and control elements, and would provide a tool for understanding the genetic basis underlying all aspects of human biology. Additionally, such a human genome discovery system would facilitate the design of primers, probes, and other reagents necessary for various genomic discovery strategies or platforms, such as nucleic acid arrays or genome re-sequencing.

The human genome comprises genes that have been associated with disease states. Many genes that have been thus identified are enumerated in the Online Mendelian Inheritance in Man (“OMIM”) database. A major goal in drug/human therapeutic development is to understand and elucidate the molecular mechanisms that govern cell signaling and cell-cell interactions in higher eukaryotes. Many disease proteins identified in the prior art have known links in cellular communication/response systems. A complete list of proteins from human that are paralogs of known human disease genes would therefore be invaluable in developing human therapeutic compounds and agents and in improving diagnostic agents and kits. Such a list of paralogous disease genes (“PDGs”) would enable better characterization of known disease genes, as well as their associated disease states. Not only will PDGs serve as models for human response, such molecules will also serve as molecular keys in differentiating pathologies caused by previously identified genes and the newly identified human paralogs. Such PDGs are likely to be commercially valuable genes for developing therapeutic agents.

Paralogous Disease Genes (PDGs)

One of ordinary skill in the art will recognize that novel paralogs of known human disease genes have many important utilities related to medicine and the development of therapeutic and diagnostic agents.

For example, paralogs, which are similar in structure, may compensate one another in function. Thus, for example, if a known human disease gene is targeted by a therapeutic agent (e.g., if expression of a gene is inhibited or reduced by antisense or RNA interference nucleic acid agents, or if the function of the encoded protein is disrupted by small molecule compounds or antibodies, etc.), the paralog of the targeted gene may compensate, thereby overcoming or reducing the effects of the therapeutic agent. In this instance, knowledge of the paralog may lead one to consider developing a supplemental therapeutic agent that targets the paralog, or developing a therapeutic agent that targets a fragment that is common between paralogs (or a fragment that is common between the proteins encoded by the parologs), so that the therapeutic agent is effective against both paralogs.

Similarly, paralogs may cross-react with therapeutic agents. In this instance, cross-reactivity of a paralog with a drug that is intended to target a different disease gene (or encoded protein) may alter the pharmacokinetics of the drug and its subsequent effectiveness. Furthermore, cross-reactivity of paralogs with therapeutic agents may lead to misleading results during drug development and clinical trials, which may consequently lead to substantial financial losses.

Additionally, paralogs may invoke previously unrecognized alternative pathways for drug activity that lead to variability in drug responses (Rothberg, Nat Biotechnol March 2001 ; 19(3):209-11).

HOX genes/Homeobox Proteins

The genes and encoded proteins provided in FIGS. 1-2 are related to HOX genes. HOX genes encode homeobox proteins, which are a family of transcription factors.

The gene and encoded protein provided in FIGS. 1A-1B match a gene/protein identified in the NCBI database as being similar to Homeobox protein Hox C12 (Hox-3F) and referred to as HOXC12 (see accession number XP_{—}171610).

The gene and encoded protein provided in FIGS. 2A-2B match a gene/protein referred to as HOXA6 (Homeobox protein A6; homeobox protein HOXA6; homeo box 1B) in the NCBI database (see accession number NP_{—}076919). For further information on HOXA6, see Kostic et al., “Targeted disruptions of the murine Hoxa-4 and Hoxa-6 genes result in homeotic transformations of components of the vertebral column”, Mech Dev June 1994; 46(3):231-47.

The genes/proteins provided in FIGS. 1-2 are paralogs of the homeobox protein IPF1 (also known as IDX1, STF1 and PDX1; NCBI accession numbers NP_{—}000200, NP_{—}037443 and NM_{—}000209, GI:4557673), mutations in which cause pancreatic agenesis (as indicated in the paralog information provided in FIGS. 1-2). For further information regarding the homeobox protein IPF1, see Stoffers et al., “Pancreatic agenesis attributable to a single nucleotide deletion in the human IPF1 gene coding sequence”, Nat Genet January 1997 ; 15(1):106-10 and Hui et al., “Pancreas duodenum homeobox-1 regulates pancreas development during embryogenesis and islet cell function in adulthood”, Eur J Endocrinol February 2002 ; 146(2):129-41.

For further information regarding HOX genes, see Goodman, “Limb malformations and the human HOX genes”, Am J Med Genet Oct. 15, 2002; 112(3):256-65 and Boncinelli, “Homeobox genes and disease”, Curr Opin Genet Dev June 1997 ; 7(3):331-7.

### SUMMARY OF THE INVENTION

The present invention is based in part on the identification of amino acid sequences, transcript sequences and gene sequences that are produced by human that show high homology to known human disease proteins. These unique protein sequences, and nucleic acid sequences that encode these proteins, can be used as models for the development of human therapeutic targets, aid in the identification of therapeutic proteins and improve diagnostic kits and reagents that are made for the duplicated gene, as well as elucidate known disease states and the role of known disease proteins therein.

### DESCRIPTION OF THE FIGURES

FIGS. 1A-1B and 2A-2B (referred to herein as FIGS. 1-2) provide PDGs of the present invention. For each PDG, FIGS. 1-2 provide the following:

1) a genomic fragment spanning the gene, including 2 KB of sequence 5′ of the identified ATG start site and 2 KB of sequence 3′ to the termination site, the predicted exon boundaries and predicted start (ATG) site, if available;

2) the predicted transcript sequence of the gene, including the starting ATG site;

3) the predicted protein sequence;

4) information related to the associated known disease gene, including gene name, disease name, accession information, and mapping information, if available.

### DETAILED DESCRIPTION OF THE INVENTION

General Description

The present invention is based on the sequencing of the human genome. During the sequencing and assembly of the human genome, analysis of the sequence information revealed previously unidentified proteins that share structural and/or sequence homology to proteins identified in the art as being involved in human disease process (human disease genes or proteins). Based on this analysis, the present invention provides amino acid sequences of these human proteins that show high homology to known human disease proteins (hereinafter the paralogous disease genes (“PDGs”) of the present invention), nucleic acid sequences that encode these PDG proteins, and information relating these PDGs/proteins to the art known human disease proteins. This information can be used by one of ordinary skill in the art to arrive at a better understanding of the biological processes underlying the diseases as well as to provide direct reagents for use in drug development.

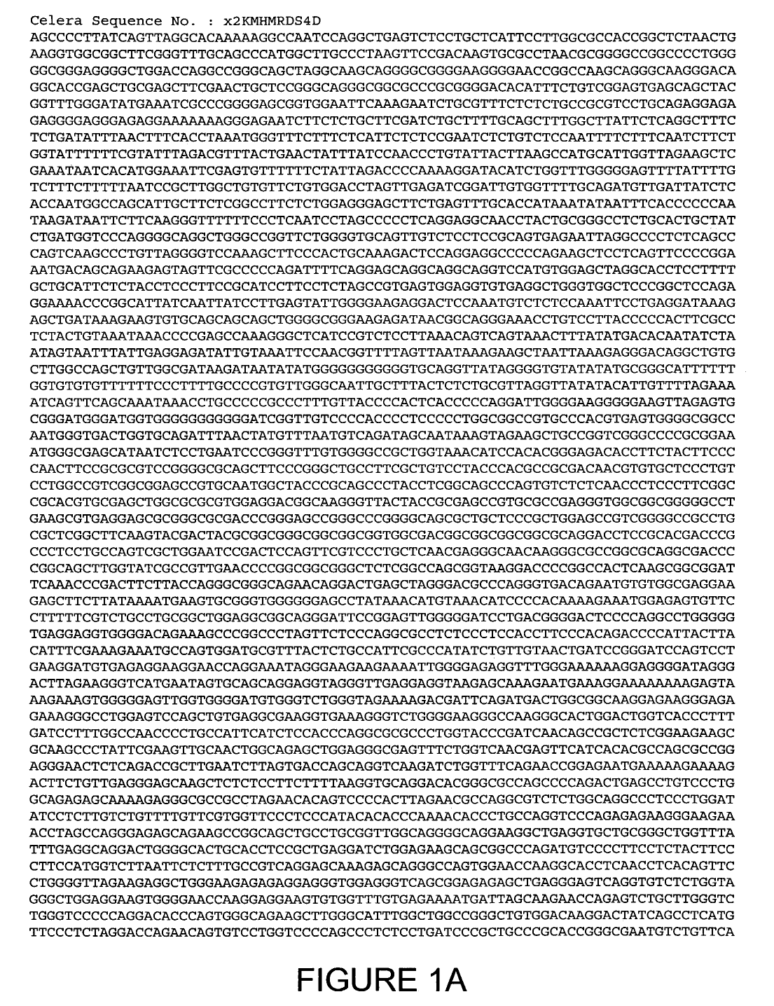

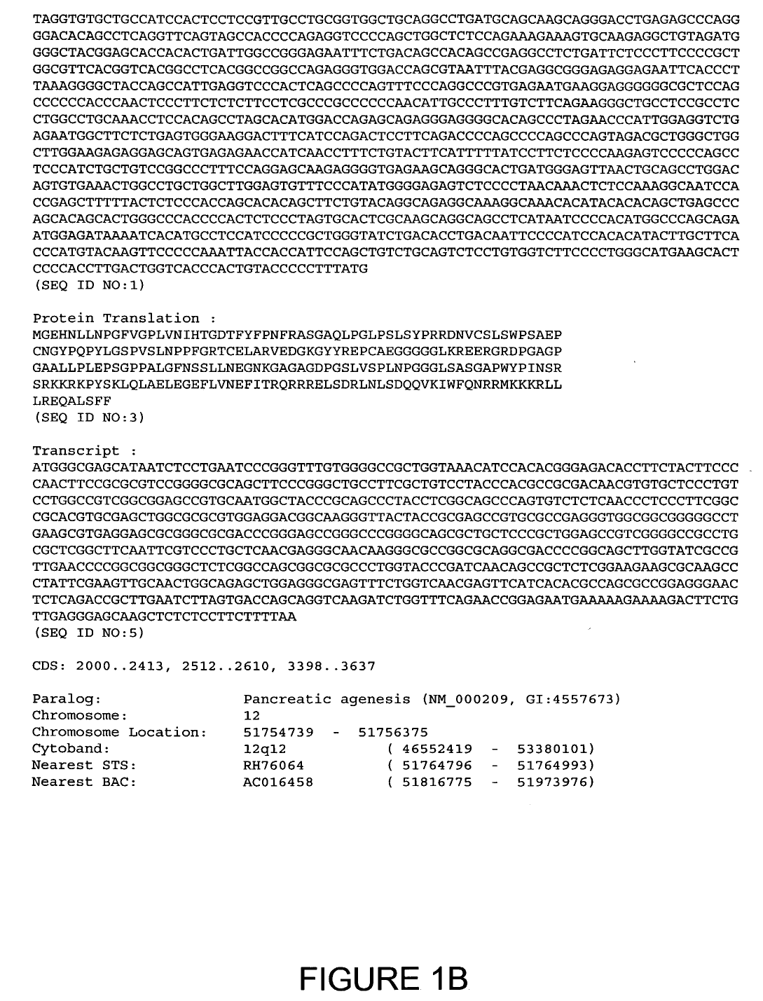

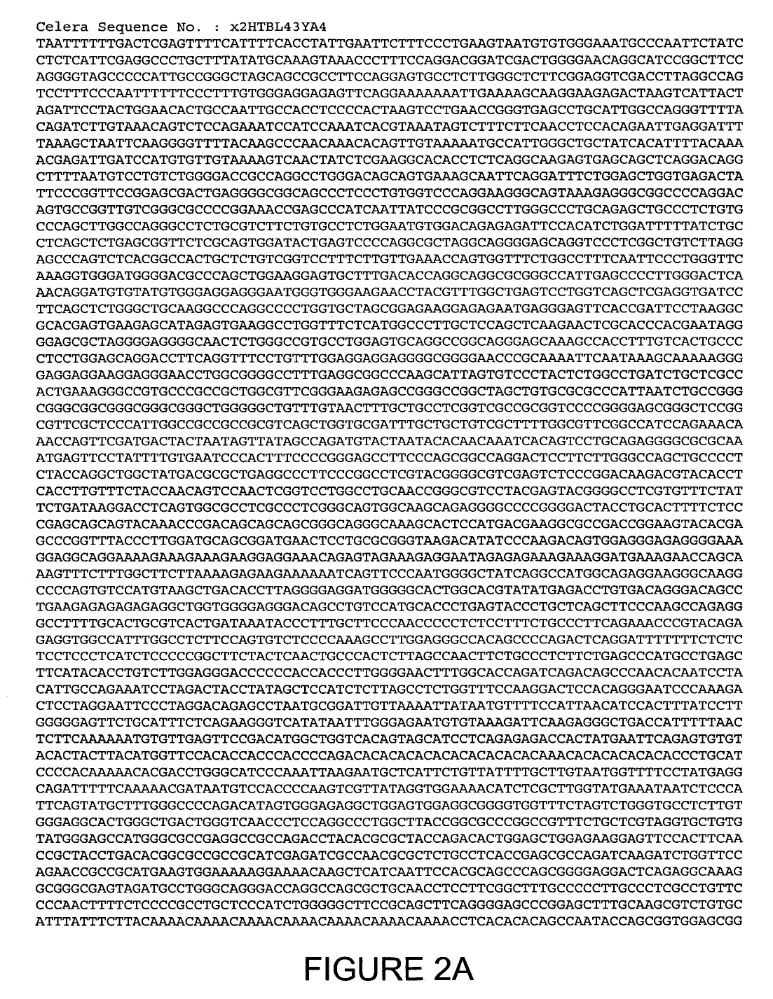

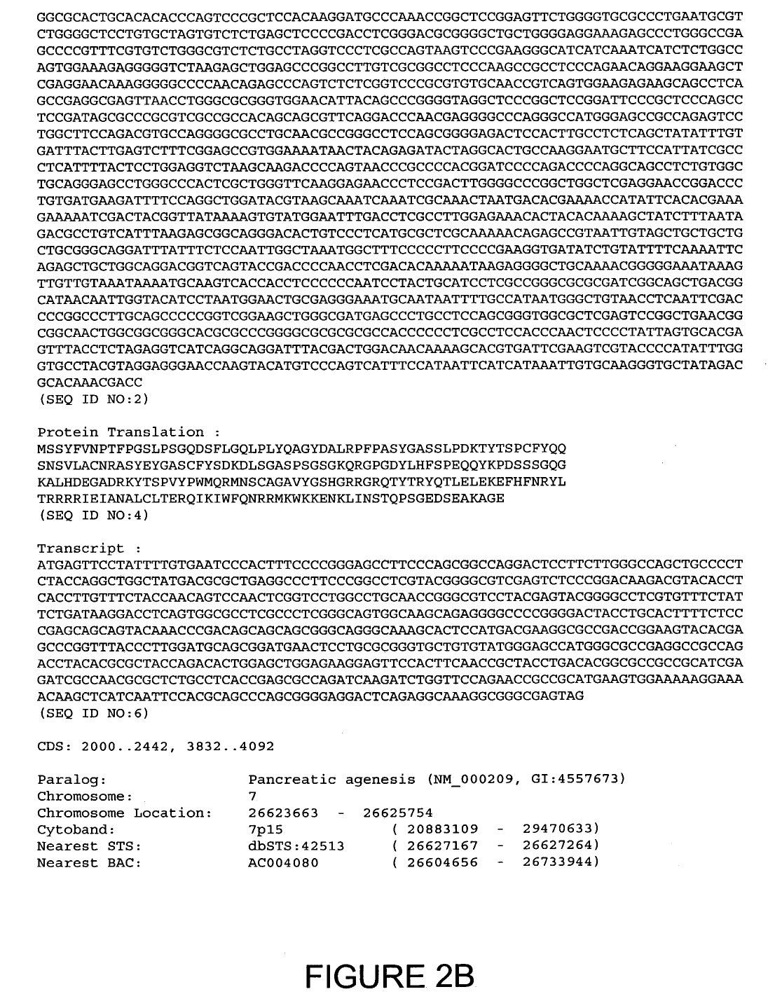

In addition to being previously unknown, the proteins that are provided in the present invention are selected based on their ability to be used for the development of commercially important products and services. Specifically, the present proteins are selected based on homology and/or structural relatedness to known human disease genes or proteins (See information contained in FIGS. 1-2). Some of the more specific features of the proteins of the present invention, and the uses thereof, are described in detail below.

Specific Embodiments

Protein Molecules

In FIGS. 1-2, the present invention provides nucleic acid molecules that encode 116 transcript and/or protein molecules (and corresponding genomic sequences) that have been identified as being encoded by genes within the human genome and show high homology to known human disease genes or proteins (paralogous disease genes (“PDGs”) of the present invention). These protein sequences will be referred herein as the PDG proteins or the PDG proteins of the present invention, or the human proteins, proteins, or peptide, genes or transcripts of the present invention.

The present invention provides isolated PDG encoded peptide and protein molecules that consist of, consist essentially of or are comprised of the amino acid sequences of the proteins encoded by the PDG encoding nucleic acid sequences disclosed in FIGS. 1-2 (the amino acid sequences are provided in SEQ ID NOS:3-4, the genomic sequences are provided in SEQ ID NOS:1-2 and the predicted transcript sequences are provided in SEQ ID NOS:5-6), as well as all obvious variants of these peptides that are within the art to make and use. Some of these variants are described in detail below.

As used herein, a protein is said to be “isolated” or “purified” when it is substantially free of cellular material or free of chemical precursors or other chemicals. The proteins of the present invention can be purified to homogeneity or other degrees of purity. The level of purification will be based on the intended use. The critical feature is that the preparation allows for the desired function of the protein, even if in the presence of considerable amounts of other components.

In some uses, “substantially free of cellular material” includes preparations of the protein having less than about 30% (by dry weight) other proteins (i.e., contaminating protein), less than about 20% other proteins, less than about 10% other proteins, or less than about 5% other proteins. When the protein is recombinantly produced, it can also be substantially free of culture medium, i.e., culture medium represents less than about 20% of the volume of the protein preparation.

The language “substantially free of chemical precursors or other chemicals” includes preparations of the protein in which it is separated from chemical precursors or other chemicals that are involved in its synthesis. In one embodiment, the language “substantially free of chemical precursors or other chemicals” includes preparations of the human protein having less than about 30% (by dry weight) chemical precursors or other chemicals, less than about 20% chemical precursors or other chemicals, less than about 10% chemical precursors or other chemicals, or less than about 5% chemical precursors or other chemicals.

The isolated PDG encoded protein can be purified from cells that naturally express it, purified from cells that have been altered to express it (recombinant), or synthesized using known protein synthesis methods. For example, a nucleic acid molecule encoding the PDG encoded protein is cloned into an expression vector, the expression vector introduced into a host cell and the protein expressed in the host cell. The protein can then be isolated from the cells by an appropriate purification scheme using standard protein purification techniques. Many of these techniques are described in detail below.

Accordingly, the present invention provides proteins that consist of one of the amino acid sequences shown in FIGS. 1-2 (the amino acid sequences are provided in SEQ ID NOS:3-4, the genomic sequences are provided in SEQ ID NOS:1-2 and the predicted transcript sequences are provided in SEQ ID NOS:5-6). The amino acid sequences of such proteins are provided in FIGS. 1-2 (the amino acid sequences are provided in SEQ ID NOS:3-4, the genomic sequences are provided in SEQ ID NOS:1-2 and the predicted transcript sequences are provided in SEQ ID NOS:5-6), along with an explanation of the protein type/family and function. A protein consists of an amino acid sequence when the amino acid sequence is the final amino acid sequence of the protein.

The present invention further provides proteins that consist essentially of one of the amino acid sequences shown in FIGS. 1-2 (the amino acid sequences are provided in SEQ ID NOS:3-4, the genomic sequences are provided in SEQ ID NOS:1-2 and the predicted transcript sequences are provided in SEQ ID NOS:5-6). A protein consists essentially of an amino acid sequence when such an amino acid sequence is present with only a few additional amino acid residues in the final protein.

The present invention further provides proteins that consist essentially of one of the amino acid sequences encoded by the nucleic acid sequences shown in FIGS. 1-2 (the amino acid sequences are provided in SEQ ID NOS:3-4, the genomic sequences are provided in SEQ ID NOS:1-2 and the predicted transcript sequences are provided in SEQ ID NOS:5-6). A protein consists essentially of an amino acid sequence when such an amino acid sequence is present with only a few additional amino acid residues in the final protein.

The present invention further provides proteins that are comprised of one of the amino acid sequences encoded by the nucleic acid sequences shown in FIGS. 1-2 (the amino acid sequences are provided in SEQ ID NOS:3-4, the genomic sequences are provided in SEQ ID NOS:1-2 and the predicted transcript sequences are provided in SEQ ID NOS:5-6). A protein is comprised of an amino acid sequence when the amino acid sequence is at least part of the final amino acid sequence of the protein. In such a fashion, the protein can be only the protein or have additional amino acid molecules, such as amino acid residues (contiguous encoded sequence) that are naturally associated with it or heterologous amino acid residues/protein sequences. Such a protein can have a few additional amino acid residues or can comprise several hundred or more additional amino acids. The preferred classes of proteins that are comprised of the PDG encoded proteins of the present invention are the naturally occurring mature proteins.

The PDG encoded proteins of the present invention can be attached to heterologous sequences to form chimeric or fusion proteins. Such chimeric and fusion proteins comprise a PDG encoded protein operatively linked to a heterologous protein having an amino acid sequence not substantially homologous to the PDG encoded protein. “Operatively linked” indicates that the PDG encoded protein and the heterologous protein are fused in-frame. The heterologous protein can be fused to the N-terminus or C-terminus of the PDG encoded protein.

In some uses, the fusion protein does not affect the activity of the PDG encoded protein per se. For example, the fusion protein can include, but is not limited to, enzymatic fusion proteins, for example beta-galactosidase fusions, yeast two-hybrid GAL fusions, poly-His fusions, MYC-tagged, HI-tagged and Ig fusions. Such fusion proteins, particularly poly-His fusions, can facilitate the purification of recombinant PDG encoded protein. In certain host cells (e.g., mammalian host cells), expression and/or secretion of a protein can be increased by using a heterologous signal sequence.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al., Current Protocols in Molecular Biology, 1992). Moreover, many expression vectors are commercially available that already encode a fusion moiety (e.g., a GST protein). A PDG nucleic acid can be cloned into such an expression vector such that the fusion moiety is linked in-frame to the PDG encoded protein.

As mentioned above, the present invention also provides and enables obvious variants of the amino acid sequence of the proteins of the present invention, such as naturally occurring mature forms of the protein, allelic/sequence variants of the proteins, non-naturally occurring recombinantly derived variants of the proteins, and orthologs and paralogs of the proteins. Such variants can readily be generated using art know techniques in the fields of recombinant nucleic acid technology and protein biochemistry. It is understood, however, that variants exclude any amino acid sequences disclosed prior to the invention.

Such variants can readily be identified/made using molecular techniques and the sequence information disclosed herein. Further, such variants can readily be distinguished from other proteins based on sequence and/or structural homology to the PDG encoded proteins of the present invention. The degree of homology/identity present will be based primarily on whether the protein is a functional variant or non-functional variant, the amount of divergence present in the paralog family and the evolutionary distance between the orthologs.

To determine the percent identity of two amino acid sequences or two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, 40%, 50%, 60%, 70%, 80%, or 90% or more of the length of the reference sequence.

The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (Devereux, J., et al, Nucleic Acids Res. 12(1):387 (1984)) (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against sequence databases to, for example, identify other family members or related sequences. Such searches can be performed using the NBLAST and XBLAST programs (version 2.0) of Altschul, et al. (J. Mol. Biol. 215:403-10 (1990)). BLAST nucleotide searches can be performed with the NBLAST program, score=100, wordlength=12 to obtain nucleotide sequences homologous to the nucleic acid molecules of the invention. BLAST protein searches can be performed with the XBLAST program, score=50, wordlength=3 to obtain amino acid sequences homologous to the proteins of the invention. To obtain gapped alignments for comparison purposes, Gapped BLAST can be utilized as described in Altschul et al. (Nucleic Acids Res. 25(17):3389-3402 (1997)). When utilizing BLAST and gapped BLAST programs, the default parameters of the respective programs (e.g., XBLAST and NBLAST) can be used. See http://www.ncbi.nlm.nih.gov.

Full-length pre-processed forms, as well as mature processed forms, of proteins that comprise one of the proteins of the present invention can readily be identified as having complete sequence identity to one of the PDG encoded proteins of the present invention as well as being encoded by the same genetic locus as the PDG encoded protein provided herein.

Allelic variants of a PDG encoded protein can readily be identified as having a high degree (significant) of sequence homology/identity to at least a portion of the PDG encoded protein as well as being encoded by the same genetic locus as the PDG encoded protein provided herein. As used herein, two proteins (or a region of the proteins) have significant homology when the amino acid sequences are typically at least about 70-75%, 80-85%, and more typically at least about 90-95% or more homologous. A significantly homologous amino acid sequence, according to the present invention, will be encoded by a nucleic acid sequence that will hybridize to a PDG encoded protein encoding nucleic acid molecule under stringent conditions as more fully described below. Further, such allelic variants will be encoded by the same genetic locus as the non-variant PDG. A skilled artisan can readily identify that a gene/protein is an allelic variant of a PDG of the present invention since the genomic position of each PDG is provided herein.

Additional paralogs of a PDG encoded protein can readily be identified as having some degree of significant sequence homology/identity to at least a portion of the PDG encoded protein, as being encoded by a gene from human, and as having similar activity or function. Two proteins will typically be considered paralogs when the amino acid sequences are typically at least about 70-75%, 80-85%, and more typically at least about 90-95% or more homologous through a given region or domain. Such paralogs will be encoded by a nucleic acid sequence that will hybridize to a PDG encoded protein encoding nucleic acid molecule under stringent conditions as more filly described below.

Orthologs of a PDG encoded protein can readily be identified as having some degree of significant sequence homology/identity to at least a portion of the PDG encoded protein as well as being encoded by a gene from another organism. Preferred orthologs will be isolated from other invertebrates. Such orthologs will be encoded by a nucleic acid sequence that will hybridize to a PDG encoded protein encoding nucleic acid molecule under moderate to stringent conditions, as more fully described below, depending on the degree of relatedness of the two organisms yielding the proteins.

Non-naturally occurring variants of the PDG encoded proteins of the present invention can readily be generated using recombinant techniques. Such variants include, but are not limited to deletions, additions and substitutions in the amino acid sequence of the PDG encoded protein. For example, one class of substitutions are conserved amino acid substitution. Such substitutions are those that substitute a given amino acid in a PDG encoded protein by another amino acid of like characteristics. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Guidance concerning which amino acid changes are likely to be phenotypically silent are found in Bowie et al., Science 247:1306-1310 (1990).

Variant PDG encoded proteins can be fully functional or can lack function in one or more activities. Fully functional variants typically contain only conservative variation or variation in non-critical residues or in non-critical regions. Functional variants can also contain substitution of similar amino acids which result in no change or an insignificant change in function. Alternatively, such substitutions may positively or negatively affect function to some degree.

Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis (Cunningham et al., Science 244:1081-1085 (1989)). The latter procedure introduces single alanine mutations at every residue in the molecule. The resulting mutant molecules are then tested for biological activity such as receptor binding or in vitro proliferative activity. Sites that are critical for ligand-receptor binding can also be determined by structural analysis such as crystallization, nuclear magnetic resonance or photoaffinity: labeling (Smith et al., J. Mol. Biol. 224:899-904 (1992); de Vos et al. Science 255:306-312 (1992)).

Proteins often contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in proteins are described in basic texts, detailed monographs, and the research literature, and they are well known to those of skill in the art.

Accordingly, the proteins also encompass derivatives or analogs in which a substituted amino acid residue is not one encoded by the genetic code, in which a substituent group is included, in which the mature protein is fused with another compound, such as a compound to increase the half-life of the protein (for example, polyethylene glycol), or in which the additional amino acids are fused to the mature protein, such as a leader or secretory sequence or a sequence for purification of the mature protein or a pro-protein sequence.

The present invention further provides fragments of the PDG encoded proteins, in addition to proteins that comprise and consist of such fragments. The fragments to which the invention pertains, however, are not to be construed as encompassing fragments that may be disclosed publicly prior to the present invention.

As used herein, a fragment comprises at least 8 or more contiguous amino acid residues from a PDG encoded protein. Such fragments can be chosen based on the ability to retain one or more of the biological activities of the PDG encoded protein or could be chosen for the ability to perform a function, e.g. act as an immunogen. Particularly important fragments are biologically active fragments, peptides that are, for example about 8 or more amino acids in length. Such fragments will typically comprise a domain or motif of the PDG encoded protein, e.g., active site. Further, possible fragments include, but are not limited to, domain or motif containing fragments, soluble peptide fragments, and fragments containing immunogenic structures. Predicted domains and functional sites are readily identifiable by computer programs well-known and readily available to those of skill in the art (e.g., PROSITE analysis).

Proteins often contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in PDG encoded proteins are described in basic texts, detailed monographs, and the research literature, and they are well known to those of skill in the art.

Accordingly, the PDG encoded proteins of the present invention also encompass derivatives or analogs in which a substituted amino acid residue is not one encoded by the genetic code, in which a substituent group is included, in which the mature PDG encoded protein is fused with another compound, such as a compound to increase the half-life of the PDG encoded protein (for example, polyethylene glycol), or in which the additional amino acids are fused to the mature PDG encoded protein, such as a leader or secretory sequence or a sequence for purification of the mature PDG encoded protein or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd Ed., T. E. Creighton, W. H. Freeman and Company, New York (1993). Many detailed reviews are available on this subject, such as by Wold, F., Post-translational Covalent Modification of Proteins, B. C. Johnson, Ed., Academic Press, New York 1-12 (1983); Seifter et al. (Meth. Enzymol. 182: 626-646 (1990)) and Rattan et al. (Ann. N.Y. Acad. Sci. 663:48-62 (1992)).

Protein/Peptide Uses

The proteins of the present invention can be used in assays to determine the biological activity of the protein, including in a panel of multiple proteins for high-throughput screening; to raise antibodies or to elicit another immune response; as a reagent (including the labeled reagent) in assays designed to quantitatively determine levels of the protein (or its binding partner or receptor) in biological fluids; and as markers for tissues in which the corresponding protein is preferentially expressed (either constitutively or at a particular stage of tissue differentiation or development or in a disease state). Where the protein binds or potentially binds to another protein (such as, for example, in a receptor-ligand interaction), the protein can be used to identify the binding partner so as to develop a system to identify inhibitors of the binding interaction. Any or all of these research utilities are capable of being developed into reagent grade or kit format for commercialization as research products.

Methods for performing the uses listed above are well known to those skilled in the art. References disclosing such methods include “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

The potential uses of the proteins of the present invention are based primarily on the source of the protein as well as the class/action of the protein. Since these PDG encoded proteins show high homology to known human disease genes/proteins, the proteins of the present invention provide valuable reagents for studying and understanding the human disease process as well as providing reagents that can be used to isolate human therapeutic products.

All of the uses for PDG encoded proteins described herein can be further used to compare and contrast a PDG to its associated known disease gene.

The PDG encoded proteins are also useful in drug/human therapeutics screening assays, in cell-based or cell-free systems. Cell-based systems can be native, i.e., cells that normally express the PDG encoded protein, as a biopsy or expanded in cell culture. In one embodiment, however, cell-based assays involve recombinant host cells expressing the PDG encoded protein. The PDG encoded proteins of the present invention are particularly useful in counter selection screens when employing the related disease gene. Such use reduces the potential for cross reactivity in drug development.

The PDG encoded proteins (including variants and fragments that may have been disclosed prior to the present invention) are useful for biological assays related to the class of proteins of which it is a member. Such assays involve any of the known protein functions or activities or properties useful for diagnosis and treatment of conditions mediated by the protein.

The proteins can be used to identify compounds that modulate the protein activity. Both the proteins of the present invention and appropriate variants and fragments can be used in high-throughput screens to assay candidate compounds for the ability to bind to the protein. These compounds can be further screened against a functional protein to determine the effect of the compound on the protein activity. Further, these compounds can be tested in animal or invertebrate systems to determine activity/effectiveness. Also, the effects of these compounds on the PDG encoded proteins of the present invention can be compared and contrasted to those on the associated known disease genes. Compounds can be identified that activate (agonist) or inactivate (antagonist) the protein to a desired degree. The PDG encoded proteins of the present invention are particularly useful in counter selection screens when employing the related disease gene. Such use reduces the potential for cross reactivity in drug development.

Further, the PDG encoded proteins can be used to screen a compound for the ability to stimulate or inhibit interaction between the PDG encoded protein and a target molecule that normally interacts with the PDG encoded protein. The target can be ligand or a component of the signal pathway that the PDG encoded protein normally interacts. Such assays typically include the steps of combining the PDG encoded protein with a candidate compound under conditions that allow the PDG encoded protein, or fragment, to interact with the target molecule, and to detect the formation of a complex between the protein and the target or to detect the biochemical consequence of the interaction with the PDG encoded protein and the target, such as any of the associated effects of signal transduction.

Candidate compounds include, for example, 1) peptides such as soluble peptides, including Ig-tailed fusion peptides and members of random peptide libraries (see, e.g., Lam et al., Nature 354:82-84 (1991); Houghten et al., Nature 354:84-86 (1991)) and combinatorial chemistry-derived molecular libraries made of D- and/or L-configuration amino acids; 2) phosphopeptides (e.g., members of random and partially degenerate, directed phosphopeptide libraries, see, e.g., Songyang et al., Cell 72:767-778 (1993)); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

One candidate compound is a soluble fragment of the PDG encoded protein that competes for ligand binding. Other candidate compounds include mutant PDG encoded proteins or appropriate fragments containing mutations that affect protein function and thus compete for ligand. Accordingly, a fragment that competes for ligand, for example with a higher affinity, or a fragment that binds ligand but does not allow release, is encompassed by the invention.

The invention further includes other end point assays to identify compounds that modulate (stimulate or inhibit) protein PDG encoded protein activity. The assays typically involve an assay of events in the signal transduction pathway that indicate PDG encoded protein activity. Thus, the expression of genes that are up- or down-regulated in response to the PDG encoded protein dependent signal cascade can be assayed. In one embodiment, the regulatory region of such genes can be operably linked to a marker that is easily detectable, such as luciferase. Alternatively, phosphorylation of the PDG encoded protein, or a PDG encoded protein target, could also be measured.

Any of the biological or biochemical functions mediated by the PDG encoded protein can be used as an endpoint assay. These include all of the biochemical or biochemical/biological events described herein, in the references cited herein, incorporated by reference for these endpoint assay targets, and other functions known to those of ordinary skill in the art.

Binding and/or activating compounds can also be screened by using chimeric PDG encoded proteins in which any of the protein's domains, or parts thereof, can be replaced by heterologous domains or subregions. Accordingly, a different set of signal transduction components is available as an end-point assay for activation. This allows for assays to be performed in other than the specific host cell from which the PDG encoded protein is derived. Additionally, the functions of the PDG encoded protein and its associated known disease gene product can be assayed by using chimeric proteins containing peptide domains or subregions from both.

The PDG encoded proteins are also useful in competition binding assays in methods designed to discover compounds that interact with the PDG encoded protein. Thus, a compound is exposed to a PDG encoded protein under conditions that allow the compound to bind or to otherwise interact with the protein. Soluble PDG encoded protein is also added to the mixture. If the test compound interacts with the soluble PDG encoded protein, it decreases the amount of complex formed or activity from the PDG encoded protein target. This type of assay is particularly useful in cases in which compounds are sought that interact with specific regions of the PDG encoded protein. Thus, the soluble protein that competes with the target PDG encoded protein region is designed to contain peptide sequences corresponding to the region of interest. Additionally, the binding characteristics of the PDG encoded protein can be compared and contrasted to those of its associated known disease gene protein.

To perform cell free drug screening assays, it is sometimes desirable to immobilize either the PDG encoded protein, or fragment, or its target molecule to facilitate separation of complexes from uncomplexed forms of one or both of the proteins, as well as to accommodate automation of the assay.

Techniques for immobilizing proteins on matrices can be used in the drug/human therapeutics screening assays. In one embodiment, a fusion protein can be provided which adds a domain that allows the protein to be bound to a matrix. For example, glutathione-S-transferase fusion proteins can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of human-binding protein found in the bead fraction quantitated from the gel using standard electrophoretic techniques. For example, either the protein or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a PDG encoded protein-binding protein and a candidate compound are incubated in the PDG encoded protein-presenting wells and the amount of complex trapped in the well can be quantitated. Methods for detecting such complexes, in addition to those described above for the GST-immobilized complexes, include immunodetection of complexes using antibodies reactive with the PDG encoded protein target molecule, or which are reactive with PDG encoded protein and compete with the target molecule, as well as enzyme-linked assays which rely on detecting an enzymatic activity associated with the target molecule.

Agents that modulate one of the PDG encoded proteins of the present invention can be identified using one or more of the above assays, alone or in combination. It is generally preferable to use a cell-based or cell free system first and then confirm activity in an animal/insect model system. Such model systems are well known in the art and can readily be employed in this context.

Modulators of PDG encoded protein activity identified according to these drug/human therapeutics screening assays can be used to treat a subject with a disorder mediated by the associated known disease gene encoded protein, by treating cells that express the human homolog. These methods of treatment include the steps of administering the modulators of protein activity in a pharmaceutical composition as described herein, to a subject in need of such treatment.

In yet another aspect of the invention, the PDG encoded proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with the PDG encoded protein and are involved in PDG encoded protein activity. Such human-binding proteins are also likely to be involved in the propagation of signals by the PDG encoded proteins or human targets as, for example, downstream elements of a human-mediated signaling pathway, e.g., a signaling pathway. Alternatively, such human-binding proteins are likely to be PDG encoded protein inhibitors.

The two-hybrid system is based on the modular nature of most transcription factors, which consist of separable DNA-binding and activation domains. Briefly, the assay utilizes two different DNA constructs. In one construct, the gene that codes for a PDG encoded protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a PDG encoded protein-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the PDG encoded protein.

This invention further pertains to novel agents identified by the above-described screening assays. Accordingly, it is within the scope of this invention to further use an agent identified as described herein in an appropriate animal model. For example, an agent identified as described herein (e.g., a PDG encoded protein modulating agent, an antisense PDG encoded protein nucleic acid molecule, a PDG encoded protein-specific antibody, or a PDG encoded protein-binding partner) can be used in an animal or insect model to determine the efficacy, toxicity, or side effects of treatment with such an agent. Furthermore, the differential effect of the PDG encoded protein and its associated known disease gene protein can be assayed. Alternatively, an agent identified as described herein can be used in an animal or insect model to determine the mechanism of action of such an agent. Furthermore, this invention pertains to uses of novel agents identified by the above-described screening assays for treatments as described herein

Antibodies

The invention also provides antibodies that selectively bind to one of the proteins of the present invention, a protein comprising such a protein, as well as variants and fragments thereof. As used herein, an antibody selectively binds a target protein when it binds the target protein and does not significantly bind to unrelated proteins. An antibody is still considered to selectively bind a protein even if it also binds to other proteins that are not substantially homologous with the target protein so long as such proteins share homology with a fragment or domain of the protein target of the antibody. In this case, it would be understood that antibody binding to the protein is still selective despite some degree of cross-reactivity, especially between the PDG encoded protein and its associated known disease gene protein. An especially useful antibody would be one that binds either the PDG encoded protein or the associated known disease gene protein preferentially to the other.

As used herein, an antibody is defined in terms consistent with that recognized within the art: they are multi-subunit proteins produced by a mammalian organism in response to an antigen challenge. The antibodies of the present invention include polyclonal antibodies and monoclonal antibodies, as well as fragments of such antibodies, including, but not limited to, Fab or F(ab′)₂, and Fv fragments.

Many methods are known for generating and/or identifying antibodies to a given target protein. Several such methods are described by Harlow, Antibodies, Cold Spring Harbor Press, (1989).

In general, to generate antibodies, an isolated peptide is used as an immunogen and is administered to a mammalian organism, such as a rat, rabbit or mouse. Either the full-length protein, an antigenic peptide fragment or a fusion protein can be used.

Antibodies are preferably prepared from regions or discrete fragments of the PDG encoded proteins. Antibodies can be prepared from any region of the protein as described herein. However, preferred regions will include those involved in function/activity and/or receptor/binding partner interaction.

An antigenic fragment will typically comprise at least 10 contiguous amino acid residues. The antigenic peptide can comprise, however, at least 12, 14, 20 or more amino acid residues. Such fragments can be selected on a physical property, such as fragments correspond to regions that are located on the surface of the protein, e.g., hydrophilic regions or can be selected based on sequence uniqueness.

Detection on an antibody of the present invention can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Uses

The antibodies can be used to isolate one of the proteins of the present invention by standard techniques, such as affinity chromatography or immunoprecipitation. The antibodies can facilitate the purification of the natural protein from cells and recombinantly produced protein expressed in host cells. In addition, such antibodies are useful to detect the presence of one of the proteins of the present invention in cells or tissues to determine the pattern of expression of the protein among various tissues in an organism and over the course of normal development. Further, such antibodies can be used to detect protein in situ, in vitro, or in a cell lysate or supernatant in order to evaluate the abundance and pattern of expression. Also, such antibodies can be used to assess abnormal tissue distribution or abnormal expression during development. Antibody detection of circulating fragments of the full length protein can be used to identify turnover.

The antibodies can also be used to assess normal and aberrant subcellular localization of cells in the various tissues in an organism. The diagnostic uses can be applied, not only in genetic testing, but also in monitoring a treatment modality. Accordingly, where treatment is ultimately aimed at correcting expression level or the presence of aberrant sequence and aberrant tissue distribution or developmental expression, antibodies directed against the or relevant fragments can be used to monitor therapeutic efficacy.

The antibodies are also useful for inhibiting protein function, for example, blocking the binding of the PDG encoded protein to a binding partner such as a receptor. These uses can also be applied in a therapeutic context in which treatment involves inhibiting the protein's function. An antibody can be used, for example, to block binding, thus modulating (agonizing or antagonizing) the proteins activity. Antibodies can be prepared against specific fragments containing sites required for function or against intact associated with a cell.

The invention also encompasses kits for using antibodies to detect the presence of a protein in a biological sample. The kit can comprise antibodies such as a labeled or labelable antibody and a compound or agent for detecting protein in a biological sample; means for determining the amount of protein in the sample; means for comparing the amount of protein in the sample with a standard; and instructions for use.

Nucleic Acid Molecules

The present invention further provides isolated nucleic acid molecules that encode a PDG encoded protein or protein of the present invention. Such nucleic acid molecules will consist of, consist essentially of, or comprise a nucleotide sequence that encodes one of the PDG encoded proteins of the present invention, an allelic variant thereof, or an ortholog or paralog thereof.

As used herein, an “isolated” nucleic acid molecule is one that is separated from other nucleic acid present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. However, there can be some flanking nucleotide sequences, for example up to about 5 KB, particularly contiguous protein encoding sequences and protein encoding sequences within the same gene but separated by introns in the genomic sequence. The important point is that the nucleic acid is isolated from remote and unimportant flanking sequences such that it can be subjected to the specific manipulations described herein such as recombinant expression, preparation of probes and primers, and other uses specific to the nucleic acid sequences.

Moreover, an “isolated” nucleic acid molecule, such as a cDNA molecule, can be substantially free of other cellular material, or culture medium when produced by recombinant techniques, or chemical precursors or other chemicals when chemically synthesized. However, the nucleic acid molecule can be fused to other coding or regulatory sequences and still be considered isolated.

For example, recombinant DNA molecules contained in a vector are considered isolated. Further examples of isolated DNA molecules include recombinant DNA molecules maintained in heterologous host cells or purified (partially or substantially) DNA molecules in solution. Isolated RNA molecules include in vivo or in vitro RNA transcripts of the isolated DNA molecules of the present invention. Isolated nucleic acid molecules according to the present invention further include such molecules produced synthetically.

Accordingly, the present invention provides nucleic acid molecules that consist of one of the nucleotide sequences shown in FIGS. 1-2 (the amino acid sequences are provided in SEQ ID NOS:3-4, the genomic sequences are provided in SEQ ID NOS:1-2 and the predicted transcript sequences are provided in SEQ ID NOS:5-6). A nucleic acid molecule consists of a nucleotide sequence when the nucleotide sequence is the complete nucleotide sequence of the nucleic acid molecule.

The present invention further provides nucleic acid molecules that consist essentially of one of the nucleotide sequences shown in FIGS. 1-2 (the amino acid sequences are provided in SEQ ID NOS:3-4, the genomic sequences are provided in SEQ ID NOS:1-2 and the predicted transcript sequences are provided in SEQ ID NOS:5-6). A nucleic acid molecule consists essentially of a nucleotide sequence when such a nucleotide sequence is present with only a few additional nucleic acid residues in the final nucleic acid molecule.

The present invention further provides nucleic acid molecules that are comprised of one of the nucleotide sequences shown in FIGS. 1-2 (the amino acid sequences are provided in SEQ ID NOS:3-4, the genomic sequences are provided in SEQ ID NOS:1-2 and the predicted transcript sequences are provided in SEQ ID NOS:5-6). A nucleic acid molecule is comprised of a nucleotide sequence when the nucleotide sequence is at least part of the final nucleotide sequence of the nucleic acid molecule. In such a fashion, the nucleic acid molecule can be only the nucleotide sequence or have additional nucleic acid residues, such as nucleic acid residues that are naturally associated with it or heterologous nucleotide sequences. Such a nucleic acid molecule can have a few additional nucleotides or can comprise several hundred or more additional nucleotides. The preferred classes of nucleic acid molecules that are comprised of the nucleotide sequences of the present are the naturally occurring full-length cDNA molecules and genes and genomic clones as provided herein.

In FIGS. 1-2, both coding and non-coding sequences are provided for each protein encoding nucleic acid sequence, for both genomic and transcript sequence when available). Because of the source of the present invention, human genomic sequences and predicted transcript sequences, the nucleic acid molecules in FIGS. 1-2 will contain genomic intronic sequences, 5′ and 3′ non-coding sequences, gene regulatory regions and non-coding intergenic sequences. In general such sequence features are either noted or can readily be identified using computational tools known in the art. As discussed below, some of the non-coding regions, particularly gene regulatory elements such as promoters, are useful for a variety of purposes, e.g. control of heterologous gene expression, target for identifying gene activity modulating compounds.

The isolated nucleic acid molecules can encode the mature protein plus additional amino or carboxyl-terminal amino acids, or amino acids interior to the mature protein (when the mature form has more than one protein chain, for instance). Such sequences may play a role in processing of a protein from precursor to a mature form, facilitate protein trafficking, prolong or shorten protein half-life or facilitate manipulation of a protein for assay or production, among other things. As generally is the case in situ, the additional amino acids may be processed away from the mature protein by cellular enzymes.

As mentioned above, the isolated nucleic acid molecules include, but are not limited to, the sequence encoding the PDG encoded protein alone, the sequence encoding the mature protein and additional coding sequences, such as a leader or secretory sequence (e.g., a pre-pro or pro-protein sequence), the sequence encoding the mature protein, with or without the additional coding sequences, plus additional non-coding sequences, for example introns and non-coding 5′ and 3′ sequences such as transcribed but non-translated sequences that play a role in transcription, mRNA processing (including splicing and polyadenylation signals), ribosome binding and stability of mRNA. In addition, the nucleic acid molecule may be fused to a marker sequence encoding, for example, a protein that facilitates purification.

Isolated nucleic acid molecules can be in the form of RNA, such as mRNA, or in the form of DNA, including cDNA and genomic DNA obtained by cloning or produced by chemical synthetic techniques or by a combination thereof. The nucleic acid, especially DNA, can be double-stranded or single-stranded. Single-stranded nucleic acid can be the coding strand (sense strand) or the non-coding strand (anti-sense strand).

The invention further provides nucleic acid molecules that encode fragments of the proteins of the present invention and that encode obvious variants of the PDG encoded proteins of the present invention that are described above. Such nucleic acid molecules may be naturally occurring, such as allelic variants (same locus), paralogs (different locus), and orthologs (different organism), or may be constructed by recombinant DNA methods or by chemical synthesis. Such non-naturally occurring variants may be made by mutagenesis techniques, including those applied to nucleic acid molecules, cells, or organisms. Accordingly, as discussed above, the variants can contain nucleotide substitutions, deletions, inversions and insertions. Variation can occur in either or both the coding and non-coding regions. The variations can produce both conservative and non-conservative amino acid substitutions.

The present invention further provides non-coding fragments of the nucleic acid molecules provided in FIGS. 1-2. Preferred non-coding fragments include, but are not limited to, promoter sequences, enhancer sequences, gene modulating sequences and gene termination sequences. Such fragments are useful in controlling heterologous gene expression and in developing screens to identify gene modulating agents. Such sequences are located 5′ to the start site identified in FIGS. 1-2 for the genomic sequences A fragment comprises a contiguous nucleotide sequence greater than 12 or more nucleotides. Further, a fragment could measure at least 30, 40, 50, 100, 250 or 500 nucleotides in length. The length of the fragment will be based on its intended use. For example, the fragment can encode epitope bearing regions of the protein, or can be useful as DNA probes and primers. Such fragments can be isolated using the known nucleotide sequence to synthesize an oligonucleotide probe. A labeled probe can then be used to screen a cDNA library, genomic DNA library, or mRNA to isolate nucleic acid corresponding to the coding region. Further, primers can be used in PCR reactions to clone specific regions of gene.

A probe/primer typically comprises substantially a purified oligonucleotide or oligonucleotide pair. The oligonucleotide typically comprises a region of nucleotide sequence that hybridizes under stringent conditions to at least about 12, 20, 25, 40, 50 or more consecutive nucleotides.

Orthologs, homologs, and allelic variants can be identified using methods well known in the art. As described in the Protein Section, these variants comprise a nucleotide sequence encoding a protein that is typically 60-65%, 70-75%, 80-85%, and more typically at least about 90-95% or more homologous to the nucleotide sequence shown in FIGS. 1-2 (the amino acid sequences are provided in SEQ ID NOS:3-4, the genomic sequences are provided in SEQ ID NOS:1-2 and the predicted transcript sequences are provided in SEQ ID NOS:5-6), or a fragment of this sequence. Such nucleic acid molecules can readily be identified as being able to hybridize under moderate to stringent conditions, to the nucleotide sequence shown in FIGS. 1-2 (the amino acid sequences are provided in SEQ ID NOS:3-4, the genomic sequences are provided in SEQ ID NOS:1-2 and the predicted transcript sequences are provided in SEQ ID NOS:5-6), or a fragment of the sequence.

As used herein, the term “hybridizes under stringent conditions” is intended to describe conditions for hybridization and washing under which nucleotide sequences encoding a protein at least 50-55% homologous to each other typically remain hybridized to each other. The conditions can be such that sequences at least about 65%, at least about 70%, or at least about 75% or more homologous to each other typically remain hybridized to each other. Such stringent conditions are known to those skilled in the art and can be found in Current Protocols in Molecular Biology, John Wiley & Sons, N.Y. (1989), 6.3.1-6.3.6. One example of stringent hybridization conditions are hybridization in 6×sodium chloride/sodium citrate (SSC) at about 45C, followed by one or more washes in 0.2×SSC, 0.1% SDS at 50-65 C.

Nucleic Acid Molecule Uses

The nucleic acid molecules of the present invention are useful for probes, primers, chemical intermediates, and in biological assays. The nucleic acid molecules are useful as a hybridization probe for cDNA and genomic DNA to isolate full-length cDNA and genomic clones encoding the protein described in FIGS. 1-2 and to isolate cDNA and genomic clones that correspond to variants (alleles, orthologs, etc.) producing the same or related proteins shown in FIGS. 1-2 and in studying the biology of known human disease proteins/genes.

The probe can correspond to any sequence along the entire length of the nucleic acid molecules provided in FIGS. 1-2 (the amino acid sequences are provided in SEQ ID NOS:3-4, the genomic sequences are provided in SEQ ID NOS:1-2 and the predicted transcript sequences are provided in SEQ ID NOS:5-6). Accordingly, it could be derived from 5′ noncoding regions, the coding region, and 3′ noncoding regions. However, as discussed, fragments are not to be construed as those which may encompass fragments disclosed prior to the present invention.

The nucleic acid molecules are also useful as primers for PCR to amplify any given region of a nucleic acid molecule and are useful to synthesize antisense molecules of desired length and sequence. Especially useful probes, primers and detection elements/reagents are those capable of differentiating a PDG from its associated known human disease gene.

The nucleic acid molecules are also useful for constructing recombinant vectors. Such vectors include expression vectors that express a portion of, or all of, the protein sequences. Vectors also include insertion vectors, used to integrate into another nucleic acid molecule sequence, such as into the cellular genome, to alter in situ expression of a gene and/or gene product. For example, an endogenous coding sequence can be replaced via homologous recombination with all or part of the coding region containing one or more specifically introduced mutations.

The nucleic acid molecules are also useful for expressing antigenic portions of the proteins.

The nucleic acid molecules are also useful as probes for determining the chromosomal positions of the nucleic acid molecules by means of in situ hybridization methods.

The nucleic acid molecules are also useful in making vectors containing the gene regulatory regions of the nucleic acid molecules of the present invention.

The nucleic acid molecules are also useful for designing ribozymes corresponding to all, or a part, of the mRNA produced from the nucleic acid molecules described herein.

The nucleic acid molecules are also useful for constructing host cells expressing a part, or all, of the nucleic acid molecules and proteins.

The nucleic acid molecules are also useful for constructing transgenic animals expressing all, or a part, of the nucleic acid molecules and proteins.

The nucleic acid molecules are also useful for making vectors that express part, or all, of the proteins.

The nucleic acid molecules are also useful as hybridization probes for determining the presence, level, form and distribution of nucleic acid expression. Accordingly, the probes can be used to detect the presence of, or to determine levels of, a specific nucleic acid molecule in cells, tissues, and in organisms. The nucleic acid whose level is determined can be DNA or RNA. Accordingly, probes corresponding to the proteins described herein can be used to assess expression and/or gene copy number in a given cell, tissue, or organism.

In vitro techniques for detection of mRNA include Northern hybridizations and in situ hybridizations. In vitro techniques for detecting DNA includes Southern hybridizations and in situ hybridization.

Probes can be used as a part of a diagnostic test kit for identifying cells or tissues that express a PDG encoded protein, such as by measuring a level of a receptor-encoding nucleic acid in a sample of cells from a subject e.g., mRNA or genomic DNA, or determining if a receptor gene has been mutated.

Nucleic acid expression assays are useful for drug/human therapeutics screening to identify compounds that modulate PDG encoded protein nucleic acid expression.

The invention thus provides a method for identifying a compound that can be used to treat a disorder associated with nucleic acid expression of the human paralog of the associate known human disease gene. The method typically includes assaying the ability of the compound to modulate the expression of the PDG encoded protein nucleic acid and thus identifying a compound that can be used to treat a disorder characterized by undesired PDG encoded protein nucleic acid expression. The assays can be performed in cell-based and cell-free systems. Cell-based assays include cells naturally expressing the PDG encoded protein nucleic acid or recombinant cells genetically engineered to express specific nucleic acid sequences.

The assay for PDG encoded protein nucleic acid expression can involve direct assay of nucleic acid levels, such as mRNA levels, or on collateral compounds involved in the signal pathway. Further, the expression of genes that are up- or down-regulated in response to the PDG encoded protein signal pathway can also be assayed. In this embodiment the regulatory regions of these genes can be operably linked to a reporter gene such as luciferase.

Thus, modulators of human gene expression can be identified in a method wherein a cell is contacted with a candidate compound and the expression of mRNA determined. The level of expression of human mRNA in the presence of the candidate compound is compared to the level of expression of human mRNA in the absence of the candidate compound. The candidate compound can then be identified as a modulator of nucleic acid expression based on this comparison and be used, for example to treat a disorder characterized by aberrant nucleic acid expression. When expression of mRNA is statistically significantly greater in the presence of the candidate compound than in its absence, the candidate compound is identified as a stimulator of nucleic acid expression. When nucleic acid expression is statistically significantly less in the presence of the candidate compound than in its absence, the candidate compound is identified as an inhibitor of nucleic acid expression.

The invention further provides methods of treatment, with the nucleic acid as a target, using a compound identified through drug/human therapeutics screening as a gene modulator to modulate the homolog of the PDG encoded protein nucleic acid expression. Modulation includes both up-regulation (i.e. activation or agonization) or down-regulation (suppression or antagonization) or nucleic acid expression.

Alternatively, a modulator for PDG encoded protein nucleic acid expression can be a small molecule or drug/human therapeutics identified using the screening assays described herein as long as the drug/human therapeutics or small molecule inhibits the PDG encoded protein nucleic acid expression.

The invention also encompasses kits for detecting the presence of a PDG encoded protein nucleic acid in a biological sample. For example, the kit can comprise reagents such as a labeled or labelable nucleic acid or agent capable of detecting PDG encoded protein nucleic acid in a biological sample; means for determining the amount of PDG encoded protein nucleic acid in the sample; and means for comparing the amount of PDG encoded protein nucleic acid in the sample with a standard. The compound or agent can be packaged in a suitable container. The kit can further comprise instructions for using the kit to detect PDG encoded protein mRNA or DNA.

Nucleic Acid Arrays

The present invention further provides arrays or microarrays of nucleic acid molecules that are based on the sequence information provided in FIGS. 1-2 (the amino acid sequences are provided in SEQ ID NOS:3-4, the genomic sequences are provided in SEQ ID NOS:1-2 and the predicted transcript sequences are provided in SEQ ID NOS:5-6).

As used herein “Arrays” or “Microarrays” refers to an array of distinct polynucleotides or oligonucleotides synthesized on a substrate, such as paper, nylon or other type of membrane, filter, chip, glass slide, or any other suitable solid support. In one embodiment, the microarray is prepared and used according to the methods described in U.S. Pat. No. 5,837,832, Chee et al., PCT application WO95/11995 (Chee et al.), Lockhart, D. J. et al. (1996; Nat. Biotech. 14: 1675-1680) and Schena, M. et al. (1996; Proc. Natl. Acad. Sci. 93: 10614-10619), all of which are incorporated herein in their entirety by reference. In other embodiments, such arrays are produced by the methods described by Brown et. al., U.S. Pat. No. 5,807,522.

The microarray is preferably composed of a large number of unique, single-stranded nucleic acid sequences, usually either synthetic antisense oligonucleotides or fragments of cDNAs, fixed to a solid support. The oligonucleotides are preferably about 6-60 nucleotides in length, more preferably 15-30 nucleotides in length, and most preferably about 20-25 nucleotides in length. For a certain type of microarray, it may be preferable to use oligonucleotides that are only 7-20 nucleotides in length. The microarray may contain oligonucleotides that cover the known 5′, or 3′, sequence, sequential oligonucleotides which cover the full length sequence; or unique oligonucleotides selected from particular areas along the length of the sequence. Polynucleotides used in the microarray may be oligonucleotides that are specific to a gene or genes of interest.

In order to produce oligonucleotides to a known sequence for a microarray, the gene(s) of interest (or an ORF identified from the contigs of the present invention) is typically examined using a computer algorithm which starts at the 5′ or at the 3′ end of the nucleotide sequence. Typical algorithms will then identify oligomers of defined length that are unique to the gene, have a GC content within a range suitable for hybridization, and lack predicted secondary structure that may interfere with hybridization. In certain situations it may be appropriate to use pairs of oligonucleotides on a microarray. The “pairs” will be identical, except for one nucleotide that preferably is located in the center of the sequence. The second oligonucleotide in the pair (mismatched by one) serves as a control. The number of oligonucleotide pairs may range from two to one million. The oligomers are synthesized at designated areas on a substrate using a light-directed chemical process. The substrate may be paper, nylon or other type of membrane, filter, chip, glass slide or any other suitable solid support.

In another aspect, an oligonucleotide may be synthesized on the surface of the substrate by using a chemical coupling procedure and an ink jet application apparatus, as described in PCT application WO95/251116 (Baldeschweiler et al.) which is incorporated herein in its entirety by reference. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures.

An array, such as those described above, may be produced by hand or by using available devices (slot blot or dot blot apparatus), materials (any suitable solid support), and machines (including robotic instruments), and may contain 8, 24, 96, 384, 1536, 6144 or more oligonucleotides, or any other number between two and one million which lends itself to the efficient use of commercially available instrumentation. In a preferred embodiment, an array contains more than one oligonucleotide. In an especially preferred embodiment, an array comprises a set of oligonucleotides derived from all 116 transcript sequences provided in SEQ ID NOS:5-6.

In order to conduct sample analysis using a microarray, the RNA or DNA from a biological sample is made into hybridization probes. The mRNA is isolated, and cDNA is produced and used as a template to make antisense RNA (aRNA). The aRNA is amplified in the presence of fluorescent nucleotides, and labeled probes are incubated with the microarray so that the probe sequences hybridize to complementary oligonucleotides of the microarray. Incubation conditions are adjusted so that hybridization occurs with precise complementary matches or with various degrees of less complementarity. After removal of nonhybridized probes, a scanner is used to determine the levels and patterns of fluorescence. The scanned images are examined to determine degree of complementarity and the relative abundance of each oligonucleotide sequence on the microarray. The biological samples may be obtained from any bodily fluids (such as blood, urine, saliva, phlegm, gastric juices, etc.), cultured cells, biopsies, or other tissue preparations. A detection system may be used to measure the absence, presence, and amount of hybridization for all of the distinct sequences simultaneously. This data may be used for large scale correlation studies on the sequences, expression patterns, mutations, variants, or polymorphisms among samples.

Using such arrays, the present invention provides methods to identify the expression of one or more of the proteins of the present invention. In detail, such methods comprise incubating a test sample with one or more nucleic acid molecules and assaying for binding of the nucleic acid molecule with components within the test sample. Such assays will typically involve arrays comprising many genes, at least one of which is a gene of the present invention.

Conditions for incubating a nucleic acid molecule with a test sample vary. Incubation conditions depend on the format employed in the assay, the detection methods employed, and the type and nature of the nucleic acid molecule used in the assay. One skilled in the art will recognize that any one of the commonly available hybridization, amplification or array assay formats can readily be adapted to employ the novel fragments of the human genome disclosed herein. Examples of such assays can be found in Chard, T, An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, Fla. Vol. 1 (1982), Vol. 2 (1983), Vol. 3 (1985); Tijssen, P., Practice and Theory of Enzyme Immunoassays: Laboratory Techniques in Biochemistry and Molecular Biology. Elsevier Science Publishers, Amsterdam, The Netherlands (1985).

The test samples of the present invention include cells, protein or membrane extracts of cells. The test sample used in the above-described method will vary based on the assay format, nature of the detection method and the tissues, cells or extracts used as the sample to be assayed. Methods for preparing nucleic acid extracts or of cells are well known in the art and can be readily be adapted in order to obtain a sample that is compatible with the system utilized.

In another embodiment of the present invention, kits are provided which contain the necessary reagents to carry out the assays of the present invention.

Specifically, the invention provides a compartmentalized kit to receive, in close confinement, one or more containers which comprises: (a) a first container comprising one of the nucleic acid molecules that can bind to a fragment of the human genome disclosed herein; and (b) one or more other containers comprising one or more of the following: wash reagents, reagents capable of detecting presence of a bound nucleic acid. Preferred kits will include chips that are capable of detecting the expression of an group of the PDG encoded protein/genes disclosed herein or the entire set.

In detail, a compartmentalized kit includes any kit in which reagents are contained in separate containers. Such containers include small glass containers, plastic containers, strips of plastic, glass or paper, or arraying material such as silica. Such containers allows one to efficiently transfer reagents from one compartment to another compartment such that the samples and reagents are not cross-contaminated, and the agents or solutions of each container can be added in a quantitative fashion from one compartment to another. Such containers will include a container which will accept the test sample, a container which contains the nucleic acid probe, containers which contain wash reagents (such as phosphate buffered saline, Tris-buffers, etc.), and containers which contain the reagents used to detect the bound probe. One skilled in the art will readily recognize that the previously unidentified genes of the present invention can be routinely identified using the sequence information disclosed herein can be readily incorporated into one of the established kit formats which are well known in the art, particularly expression arrays.

Vectors/Host Cells

The invention also provides vectors containing the nucleic acid molecules described herein. The term “vector” refers to a vehicle, preferably a nucleic acid molecule, that can transport the nucleic acid molecules. When the vector is a nucleic acid molecule, the nucleic acid molecules are covalently linked to the vector nucleic acid. With this aspect of the invention, the vector includes a plasmid, single or double stranded phage, a single or double stranded RNA or DNA viral vector, or artificial chromosome, such as a BAC, PAC, YAC, OR MAC.

A vector can be maintained in the host cell as an extrachromosomal element where it replicates and produces additional copies of the nucleic acid molecules. Alternatively, the vector may integrate into the host cell genome and produce additional copies of the nucleic acid molecules when the host cell replicates.

The invention provides vectors for the maintenance (cloning vectors) or vectors for expression (expression vectors) of the nucleic acid molecules. The vectors can function in prokaryotic or eukaryotic cells or in both (shuttle vectors).

Expression vectors contain cis-acting regulatory regions that are operably linked in the vector to the nucleic acid molecules such that transcription of the nucleic acid molecules is allowed in a host cell. The nucleic acid molecules can be introduced into the host cell with a separate nucleic acid molecule capable of affecting transcription. Thus, the second nucleic acid molecule may provide a trans-acting factor interacting with the cis-regulatory control region to allow transcription of the nucleic acid molecules from the vector. Alternatively, a trans-acting factor may be supplied by the host cell. Finally, a trans-acting factor can be produced from the vector itself. It is understood, however, that in some embodiments, transcription and/or translation of the nucleic acid molecules can occur in a cell-free system.

The regulatory sequence to which the nucleic acid molecules described herein can be operably linked include promoters for directing mRNA transcription. These include, but are not limited to, the left promoter from bacteriophage λ, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

In addition to containing sites for transcription initiation and control, expression vectors can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al., Molecular Cloning: A Laboratory Manual. 2nd. ed., Cold Spring Harbor Laboratory Press, Cold Spring Harbor, N.Y., (1989).

A variety of expression vectors can be used to express a nucleic acid molecule. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, poxviruses, pseudorabies viruses, and retroviruses. Vectors may also be derived from combinations of these sources such as those derived from plasmid and bacteriophage genetic elements, eg. cosmids and phagemids. Appropriate cloning and expression vectors for prokaryotic and eukaryotic hosts are described in Sambrook et al., Molecular Cloning: A Laboratory Manual. 2nd. ed., Cold Spring Harbor Laboratory Press, Cold Spring Harbor, N.Y., (1989).

The regulatory sequence may provide constitutive expression in one or more host cells (i.e. tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

The nucleic acid molecules can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art.

The vector containing the appropriate nucleic acid molecule can be introduced into an appropriate host cell for propagation or expression using well-known techniques. Bacterial cells include, but are not limited to, E. coli, Streptomyces, and Salmonella typhimurium. Eukaryotic cells include, but are not limited to, yeast, insect cells such as human, animal cells such as COS and CHO cells, and plant cells.

As described herein, it may be desirable to express the protein as a fusion protein. Accordingly, the invention provides fusion vectors that allow for the production of the proteins. Fusion vectors can increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired peptide can ultimately be separated from the fusion moiety. Proteolytic enzymes include, but are not limited to, factor Xa, and thrombin. Typical fusion expression vectors include pGEX (Smith et al., Gene 67:31-40 (1988)), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al., Gene 69:301-315 (1988)) and pET 11d (Studier et al., Gene Expression Technology: Methods in Enzymology 185:60-89 (1990)).

Recombinant protein expression can be maximized in a host bacteria by providing a genetic background wherein the host cell has an impaired capacity to proteolytically cleave the recombinant protein. (Gottesman, S., Gene Expression Technology: Methods in Enzymology 185, Academic Press, San Diego, Calif. (1990) 119-128). Alternatively, the sequence of the nucleic acid molecule of interest can be altered to provide preferential codon usage for a specific host cell, for example E. coli. (Wada et al., Nucleic Acids Res. 20:2111-2118 (1992)).

The nucleic acid molecules can also be expressed by expression vectors that are operative in yeast. Examples of vectors for expression in yeast e.g., S. cerevisiae include pYepSec1 (Baldari, et al., EMBO J. 6:229-234 (1987)), pMFa (Kurjan et al., Cell 30:933-943(1982)), pJRY88 (Schultz et al., Gene 54:113-123 (1987)), and pYES2 (Invitrogen Corporation, San Diego, Calif.).

The nucleic acid molecules can also be expressed in insect cells using, for example, baculovirus expression vectors. Baculovirus vectors available for expression of proteins in cultured insect cells (e.g., Sf9 cells) include the pAc series (Smith et al., Mol. Cell Biol. 3:2156-2165 (1983)) and the pVL series (Lucklow et al., Virology 170:31-39 (1989)).

In certain embodiments of the invention, the nucleic acid molecules described herein are expressed in mammalian cells using mammalian expression vectors. Examples of mammalian expression vectors include pCDM8 (Seed, B. Nature 329:840(1987)) and pMT2PC (Kaufman et al., EMBO J. 6:187-195 (1987)).

The expression vectors listed herein are provided by way of example only of the well-known vectors available to those of ordinary skill in the art that would be useful to express the nucleic acid molecules. The person of ordinary skill in the art would be aware of other vectors suitable for maintenance propagation or expression of the nucleic acid molecules described herein. These are found for example in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

The invention also encompasses vectors in which the nucleic acid sequences described herein are cloned into the vector in reverse orientation, but operably linked to a regulatory sequence that permits transcription of antisense RNA. Thus, an antisense transcript can be produced to all, or to a portion, of the nucleic acid molecule sequences described herein, including both coding and non-coding regions. Expression of this antisense RNA is subject to each of the parameters described above in relation to expression of the sense RNA (regulatory sequences, constitutive or inducible expression, tissue-specific expression).

The invention also relates to recombinant host cells containing the vectors described herein. Host cells therefore include prokaryotic cells, lower eukaryotic cells such as yeast, other eukaryotic cells such as insect cells, and higher eukaryotic cells such as mammalian cells.

The recombinant host cells are prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989).

Host cells can contain more than one vector. Thus, different nucleotide sequences can be introduced on different vectors of the same cell. Similarly, the nucleic acid molecules can be introduced either alone or with other nucleic acid molecules that are not related to the nucleic acid molecules such as those providing trans-acting factors for expression vectors. When more than one vector is introduced into a cell, the vectors can be introduced independently, co-introduced or joined to the nucleic acid molecule vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Vectors generally include selectable markers that enable the selection of the sub-population of cells that contain the recombinant vector constructs. The marker can be contained in the same vector that contains the nucleic acid molecules described herein or may be on a separate vector. Markers include tetracycline or ampicillin-resistance genes for prokaryotic host cells and dihydrofolate reductase or neomycin resistance for eukaryotic host cells. However, any marker that provides selection for a phenotypic trait will be effective.

While the mature proteins can be produced in bacteria, yeast, mammalian cells, and other cells under the control of the appropriate regulatory sequences, cell-free transcription and translation systems can also be used to produce these proteins using RNA derived from the DNA constructs described herein.

Where secretion of the peptide is desired, appropriate secretion signals are incorporated into the vector. The signal sequence can be endogenous to the peptides or heterologous to these peptides.

Where the peptide is not secreted into the medium, the protein can be isolated from the host cell by standard disruption procedures, including freeze thaw, sonication, mechanical disruption, use of lysing agents and the like. The peptide can then be recovered and purified by well-known purification methods including ammonium sulfate precipitation, acid extraction, anion or cationic exchange chromatography, phosphocellulose chromatography, hydrophobic-interaction chromatography, affinity chromatography, hydroxylapatite chromatography, lectin chromatography, or high performance liquid chromatography.

It is also understood that depending upon the host cell in recombinant production of the peptides described herein, the peptides can have various glycosylation patterns, depending upon the cell, or may be non-glycosylated as when produced in bacteria. In addition, the peptides may include an initial modified methionine in some cases as a result of a host-mediated process.

Uses of Vectors and Host Cells

The recombinant host cells expressing the proteins described herein have a variety of uses. First, the cells are useful for producing a PDG encoded protein or protein that can be further purified to produce desired amounts of PDG encoded protein or fragments. Thus, host cells containing expression vectors are useful for protein production.

Host cells are also useful for conducting cell-based assays involving the PDG encoded protein or PDG encoded protein fragments. Thus, a recombinant host cell expressing a native PDG encoded protein is useful for assaying compounds that stimulate or inhibit PDG encoded protein function.

Host cells are also useful for identifying PDG encoded protein mutants in which these functions are affected. If the mutants naturally occur and give rise to a pathology, host cells containing the mutations are useful to assay compounds that have a desired effect on the mutant PDG encoded protein (for example, stimulating or inhibiting function) which may not be indicated by their effect on the native PDG encoded protein.

Genetically engineered host cells can be further used to produce non-human transgenic animals. A transgenic animal is preferably a mammal, for example a rodent, such as a rat or mouse, in which one or more of the cells of the animal include a transgene. A transgene is exogenous DNA which is integrated into the genome of a cell from which a transgenic animal develops and which remains in the genome of the mature animal in one or more cell types or tissues of the transgenic animal. These animals are useful for studying the function of a PDG encoded protein and identifying and evaluating modulators of PDG encoded protein activity. Other examples of transgenic animals include non-human primates, sheep, dogs, cows, goats, chickens, and amphibians.

A transgenic animal can be produced by introducing nucleic acid into the male pronuclei of a fertilized oocyte, e.g., by microinjection, retroviral infection, and allowing the oocyte to develop in a pseudopregnant female foster animal. Any of the PDG encoded protein nucleotide sequences can be introduced as a transgene into the genome of a non-human animal, such as a mouse.

Any of the regulatory or other sequences useful in expression vectors can form part of the transgenic sequence. This includes intronic sequences and polyadenylation signals, if not already included. A tissue-specific regulatory sequence(s) can be operably linked to the transgene to direct expression of the PDG encoded protein to particular cells.

Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes. A transgenic animal also includes animals in which the entire animal or tissues in the animal have been produced using the homologously recombinant host cells described herein.

Clones of the non-human transgenic animals described herein can also be produced according to the methods described in Wilmut, I. et al. Nature 385:810-813 (1997) and PCT International Publication Nos. WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring born of this female foster animal will be a clone of the animal from which the cell, e.g., the somatic cell, is isolated.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

That which is claimed is:

1. An isolated polypeptide consisting of an amino acid sequence selected from the group consisting of the amino acid sequence shown in FIGS. 1-2, SEQ ID NOS:3-4;

2. An isolated polypeptide comprising an amino acid sequence selected from the group consisting of the amino acid sequence shown in FIGS. 1-2, SEQ ID NOS:3-4;

3. An isolated antibody that selectively binds to the polypeptide of claim 1.

4. An isolated nucleic acid molecule consisting of a nucleotide sequence selected from the group consisting of:
(a) a nucleotide sequence that encodes an amino acid sequence shown in FIGS. 1-2, SEQ ID NOS:3-4;
(b) a transcript sequence shown in FIGS. 1-2, SEQ ID NOS:5-6;
(c) a genomic sequence shown in FIGS. 1-2, SEQ ID NOS:1-2; and
(d) a nucleic acid molecule that is completely complementary to a nucleic acid molecule of (a)-(c).

5. An isolated nucleic acid molecule comprising a nucleotide sequence selected from the group consisting of:
(a) a nucleotide sequence that encodes an amino acid sequence shown in FIGS. 1-2, SEQ ID NOS:3-4;
(b) a transcript sequence shown in FIGS. 1-2, SEQ ID NOS:5-6;
(c) a genomic sequence shown in FIGS. 1-2, SEQ ID NOS:1-2; and
(e) (d) a nucleic acid molecule that is completely complementary to a nucleic acid molecule of (a)-(c).

6. A nucleic acid array comprising the nucleic acid molecule of claim 4.

7. A transgenic non-human animal comprising the nucleic acid molecule of claim 4.

8. A vector comprising the nucleic acid molecule of claim 4.

9. A host cell containing the vector of claim 8.

10. A method for producing the polypeptide of claim 1, the method comprising introducing a nucleotide sequence encoding the polypeptide of claim 1 into a host cell, and culturing the host cell under conditions in which the polypeptide is expressed from the nucleic acid.

11. A method for detecting the presence of the polypeptide of claim 1 in a sample, said method comprising contacting said sample with an agent that specifically allows detection of the presence of the polypeptide in the sample, and detecting the presence of the polypeptide.

12. A kit comprising reagents used for the method of claim 11, wherein the reagents comprise an agent that specifically binds to said polypeptide.

13. A method for detecting the presence of the nucleic acid molecule of claim 4 in a sample, said method comprising contacting the sample with an oligonucleotide comprising at least about 20 contiguous nucleotides that hybridizes to said nucleic acid molecule under stringent conditions, wherein said stringent conditions are hybridization in 6×sodium chloride/sodium citrate (SSC) at about 45° C., followed by one or more washes in 0.2×SCC, 0.1% SDS at 50-65° C., and determining whether the oligonucleotide binds to said nucleic acid molecule.

14. A method for identifying a modulator of the polypeptide of claim 1, said method comprising contacting said polypeptide, or contacting a cell expressing said polypeptide, with an agent and determining if said agent has modulated the function, activity, or expression of said polypeptide.

15. The method of claim 14, wherein said agent is administered to a host cell comprising an expression vector that expresses said polypeptide.

16. A method for identifying an agent that binds to the polypeptides of claim 1, said method comprising contacting the polypeptide with an agent and assaying the contacted mixture to determine whether a complex is formed with the agent bound to the polypeptide.

17. A pharmaceutical composition comprising an agent identified by the method of claim 16 and a pharmaceutically acceptable carrier therefor.

18. A method for treating a disease or condition, said method comprising administering to a patient a pharmaceutically effective amount of an agent identified by the method of claim 16.

19. An isolated human polypeptide having an amino acid sequence that shares at least 99% sequence identity with the polypeptide of claim 1.

20. An isolated nucleic acid molecule encoding a polypeptide, said nucleic acid molecule sharing at least 99% sequence identity with the nucleic acid molecule of claim 4.


SEQUENCE LISTING (6 sequences)

SEQ ID NO: 1 - DNA, 5638 bp, Homo sapiens
agccccttatcagttaggcacaaaaaggccaatccaggctgagtctcctgctcattccttggcgccaccggctctaactgaaggtggcggcttcgggtttgcagcccatggcttgccctaagttccgacaagtgcgcctaacgcggggccggcccctgggggcgggaggggctggaccaggccgggcagctaggcaagcaggggcggggaaggggaaccggccaagcagggcaagggacaggcaccgagctgcgagcttcgaactgctccgggcagggcggcgcccgcggggacacatttctgtcggagtgagcagctacggtttgggatatgaaatcgcccggggagcggtggaattcaaagaatctgcgtttctctctgccgcgtcctgcagaggagagaggggagggagaggaaaaaaagggagaatcttctctgcttcgatctgcttttgcagctttggcttattctcaggctttctctgatatttaactttcacctaaatgggtttctttctcattctctccgaatctctgtctccaattttctttcaatcttctggtattttttcgtatttagacgtttactgaactatttatccaaccctgtattacttaagccatgcattggttagaagctcgaaataatcacatggaaattcgagtgttttttctattagaccccaaaaggatacatctggtttgggggagttttattttgtctttctttttaatccgcttggctgtgttctgtggacctagttgagatcggattgtggttttgcagatgttgattatctcaccaatggccagcattgcttctcggccttctctggagggagcttctgagtttgcaccataaatataatttcaccccccaataagataattcttcaagggttttttccctcaatcctagccccctcaggaggcaacctactgcgggcctctgcactgctatctgatggtcccaggggcaggctgggccggttctggggtgcagttgtctcctccgcagtgagaattaggcccctctcagcccagtcaagccctgttaggggtccaaagcttcccactgcaaagactccaggaggcccccagaagctcctcagttccccggaaatgacagcagaagagtagttcgcccccagattttcaggagcaggcaggcaggtccatgtggagctaggcacctccttttgctgcattctctacctcccttccgcatccttcctctagccgtgagtggaggtgtgaggctgggtggctcccggctccagaggaaaacccggcattatcaattatccttgagtattggggaagaggactccaaatgtctctccaaattcctgaggataaagagctgataaagaagtgtgcagcagcagctggggcgggaagagataacggcagggaaacctgtccttacccccacttcgcctctactgtaaataaaccccgagccaaagggctcatccgtctccttaaacagtcagtaaactttatatgacacaatatctaatagtaatttattgaggagatattgtaaattccaacggttttagttaataaagaagctaattaaagagggacaggctgtgcttggccagctgttggcgataagataatatatgggggggggggtgcaggttataggggtgtatatatgcgggcattttttggtgtgtgttttttcccttttgccccgtgttgggcaattgctttactctctgcgttaggttatatacattgttttagaaaatcagttcagcaaataaacctgcccccgccctttgttaccccactcacccccaggattggggaagggggaagttagagtgcgggatgggatggtgggggggggggatcggttgtccccacccctccccctggcggccgtgcccacgtgagtggggcggccaatgggtgactggtgcagatttaactatgtttaatgtcagatagcaataaagtagaagctgccggtcgggccccgcggaaatgggcgagcataatctcctgaatcccgggtttgtggggccgctggtaaacatccacacgggagacaccttctacttccccaacttccgcgcgtccggggcgcagcttcccgggctgccttcgctgtcctacccacgccgcgacaacgtgtgctccctgtcctggccgtcggcggagccgtgcaatggctacccgcagccctacctcggcagcccagtgtctctcaaccctcccttcggccgcacgtgcgagctggcgcgcgtggaggacggcaagggttactaccgcgagccgtgcgccgagggtggcggcgggggcctgaagcgtgaggagcgcgggcgcgacccgggagccgggcccggggcagcgctgctcccgctggagccgtcggggccgcctgcgctcggcttcaagtacgactacgcggcgggcggcggcggtggcgacggcggcggcggcgcaggacctccgcacgacccgccctcctgccagtcgctggaatccgactccagttcgtccctgctcaacgagggcaacaagggcgccggcgcaggcgaccccggcagcttggtatcgccgttgaaccccggcggcgggctctcggccagcggtaaggaccccggccactcaagcggcggattcaaacccgacttcttaccagggcgggcagaacaggactgagctagggacgcccagggtgacagaatgtgtggcgaggaagagcttcttataaaatgaagtgcgggtggggggagcctataaacatgtaaacatccccacaaaagaaatggagagtgttcctttttcgtctgcctgcggctggaggcggcagggattccggagttgggggatcctgacggggactccccaggcctgggggtgaggaggtggggacagaaagcccggccctagttctcccaggcgcctctccctccaccttcccacagaccccattacttacatttcgaaagaaatgccagtggatgcgtttactctgccattcgcccatatctgttgtaactgatccgggatccagtcctgaaggatgtgagaggaaggaaccaggaaatagggaagaagaaaattggggagaggtttgggaaaaaaggaggggatagggacttagaagggtcatgaatagtgcagcaggaggtagggttgaggaggtaagagcaaagaatgaaaggaaaaaaaagagtaaagaaagtgggggagttggtggggatgtgggtctgggtagaaaagacgattcagatgactggcggcaaggagaagggagagaaagggcctggagtccagctgtgaggcgaaggtgaaagggtctggggaagggccaagggcactggactggtcaccctttgatcctttggccaacccctgccattcatctccacccaggcgcgccctggtacccgatcaacagccgctctcggaagaagcgcaagccctattcgaagttgcaactggcagagctggagggcgagtttctggtcaacgagttcatcacacgccagcgccggagggaactctcagaccgcttgaatcttagtgaccagcaggtcaagatctggtttcagaaccggagaatgaaaaagaaaagacttctgttgagggagcaagctctctccttcttttaaggtgcaggacacgggcgccagccccagactgagcctgtccctggcagagagcaaaagagggcgccgcctagaacacagtccccacttagaacgccaggcgtctctggcaggccctccctggatatcctcttgtctgttttgttcgtggttccctcccatacacacccaaaacaccctgccaggtcccagagagaagggaagaaacctagccagggagagcagaagccggcagctgcctgcggttggcaggggcaggaaggctgaggtgctgcgggctggtttatttgaggcaggactggggcactgcacctccgctgaggatctggagaagcagcggcccagatgtccccttcctctacttcccttccatggtcttaattctctttgccgtcaggagcaaagagcagggccagtggaaccaaggcacctcaacctcacagttcctggggttagaagaggctgggaagagagaggagggtggagggtcagcggagagagctgagggagtcaggtgtctctggtagggctggaggaagtggggaaccaaggaggaagtgtggtttgtgagaaaatgattagcaagaaccagagtctgcttgggtctgggtcccccaggacacccagtgggcagaagcttgggcatttggctggccgggctgtggacaaggactatcagcctcatgttccctctaggaccagaacagtgtcctggtccccagccctctcctgatcccgctgcccgcaccgggcgaatgtctgttcataggtgtgctgccatccactcctccgttgcctgcggtggctgcaggcctgatgcagcaagcagggacctgagagcccaggggacacagcctcaggttcagtagccaccccagaggtccccagctggctctccagaaagaaagtgcaagaggctgtagatggggctacggagcaccacactgattggccgggagaatttctgacagccacagccgaggcctctgattctcccttccccgctggcgttcacggtcacggcctcacggccggccagagggtggaccagcgtaatttacgaggcgggagaggagaattcacccttaaaggggctaccagccattgaggtcccactcagccccagtttcccaggcccgtgagaatgaaggaggggggcgctccagccccccacccaactcccttctctcttcctcgcccgccccccaacattgccctttgtcttcagaagggctgcctccgcctcctggcctgcaaacctccacagcctagcacatggaccagagcagagggaggggcacagccctagaacccattggaggtctgagaatggcttctctgagtgggaaggactttcatccagactccttcagaccccagccccagcccagtagacgctgggctggcttggaagagaggagcagtgagagaaccatcaacctttctgtacttcatttttatccttctccccaagagtcccccagcctcccatctgctgtccggccctttccaggagcaagaggggtgagaagcagggcactgatgggagttaactgcagcctggacagtgtgaaactggcctgctggcttggagtgtttcccatatggggagagtctcccctaacaaactctccaaaggcaatccaccgagctttttactctcccaccagcacacagcttctgtacaggcagaggcaaaggcaaacacatacacacagctgagcccagcacagcactgggcccaccccactctccctagtgcactcgcaagcaggcagcctcataatccccacatggcccagcagaatggagataaaatcacatgcctccatcccccgctgggtatctgacacctgacaattccccatccacacatacttgcttcacccatgtacaagttcccccaaattaccaccattccagctgtctgcagtctcctgtggtcttcccctgggcatgaagcactccccaccttgactggtcacccactgtaccccctttatg

SEQ ID NO: 2 - DNA, 6092 bp, Homo sapiens
taattttttgactcgagttttcattttcacctattgaattctttccctgaagtaatgtgtgggaaatgcccaattctatcctctcattcgaggccctgctttatatgcaaagtaaaccctttccaggacggatcgactggggaacaggcatccggcttccaggggtagcccccattgccgggctagcagccgccttccaggagtgcctcttgggctcttcggaggtcgaccttaggccagtcctttcccaattttttccctttgtgggaggagagttcaggaaaaaaattgaaaagcaaggaagagactaagtcattactagattcctactggaacactgccaattgccacctccccactaagtcctgaaccgggtgagcctgcattggccagggttttacagatcttgtaaacagtctccagaaatccatccaaatcacgtaaatagtctttcttcaacctccacagaattgaggattttaaagctaattcaaggggttttacaagcccaacaaacacagttgtaaaaatgccattgggctgctatcacattttacaaaacgagattgatccatgtgttgtaaaagtcaactatctcgaaggcacacctctcaggcaagagtgagcagctcaggacaggcttttaatgtcctgtctggggaccgccaggcctgggacagcagtgaaagcaattcaggatttctggagctggtgagactattcccggttccggagcgactgaggggcggcagccctccctgtggtcccaggaagggcagtaaagagggcggccccaggacagtgccggttgtcgggcgccccggaaaccgagcccatcaattatcccgcggccttgggccctgcagagctgccctctgtgcccagcttggccagggcctctgcgtcttctgtgcctctggaatgtggacagagagattccacatctggatttttatctgcctcagctctgagcggttctcgcagtggatactgagtccccaggcgctaggcaggggagcaggtccctcggctgtcttaggagcccagtctcacggccactgctctgtcggtcctttcttgttgaaaccagtggtttctggcctttcaattccctgggttcaaaggtgggatggggacgcccagctggaaggagtgctttgacaccaggcaggcgcgggccattgagccccttgggactcaaacaggatgtgtatgtgggaggagggaatgggtgggaagaacctacgtttggctgagtcctggtcagctcgaggtgatccttcagctctgggctgcaaggcccaggcccctggtgctagcggagaaggagagaatgagggagttcaccgattcctaaggcgcacgagtgaagagcatagagtgaaggcctggtttctcatggcccttgctccagctcaagaactcgcacccacgaataggggagcgctaggggaggggcaactctgggccgtgcctggagtgcaggccggcagggagcaaagccacctttgtcactgcccctcctggagcaggaccttcaggtttcctgtttggaggaggaggggcggggaacccgcaaaattcaataaagcaaaaaggggaggaggaaggagggaacctggcggggcctttgaggcggcccaagcattagtgtccctactctggcctgatctgctcgccactgaaagggccgtgcccgccgctggcgttcgggaagagagccgggccggctagctgtgcgcgcccattaatctgccgggcgggcggcgggcgggcgggctgggggctgtttgtaactttgctgcctcggtcgccgcggtccccggggagcgggctccggcgttcgctcccattggccgccgccgcgtcagctggtgcgatttgctgctgtcgcttttggcgttcggccatccagaaacaaaccagttcgatgactactaatagttatagccagatgtactaatacacaacaaatcacagtcctgcagaggggcgcgcaaatgagttcctattttgtgaatcccactttccccgggagccttcccagcggccaggactccttcttgggccagctgcccctctaccaggctggctatgacgcgctgaggcccttcccggcctcgtacggggcgtcgagtctcccggacaagacgtacacctcaccttgtttctaccaacagtccaactcggtcctggcctgcaaccgggcgtcctacgagtacggggcctcgtgtttctattctgataaggacctcagtggcgcctcgccctcgggcagtggcaagcagaggggccccggggactacctgcacttttctcccgagcagcagtacaaacccgacagcagcagcgggcagggcaaagcactccatgacgaaggcgccgaccggaagtacacgagcccggtttacccttggatgcagcggatgaactcctgcgcgggtaagacatatcccaagacagtggagggagaggggaaaggaggcaggaaaagaaagaaagaaggaggaaacagagtagaaagaggaatagagagaaagaaaggatgaaagaaccagcaaagtttctttggcttcttaaaagagaagaaaaaatcagttcccaatggggctatcaggccatggcagaggaagggcaaggccccagtgtccatgtaagctgacaccttaggggaggatgggggcactggcacgtatatgagacctgtgacagggacagcctgaagagagagagaggctggtggggagggacagcctgtccatgcaccctgagtaccctgctcagcttcccaagccagagggccttttgcactgcgtcactgataaataccctttgcttcccaaccccctctcctttctgcccttcagaaacccgtacagagaggtggccatttggcctcttccagtgtctccccaaagccttggagggccacagccccagactcaggatttttttctctctcctccctcatctcccccggcttctactcaactgcccactcttagccaacttctgccctcttctgagcccatgcctgagcttcatacacctgtcttggagggaccccccaccacccttggggaactttggcaccagatcagacagcccaacacaatcctacattgccagaaatcctagactacctatagctccatctcttagcctctggtttccaaggactccacagggaatcccaaagactcctaggaattccctaggacagagcctaatgcggattgttaaaattataatgttttccattaacatccactttatccttgggggagttctgcatttctcagaagggtcatataatttgggagaatgtgtaaagattcaagagggctgaccatttttaactcttcaaaaaatgtgttgagttccgacatggctggtcacagtagcatcctcagagagaccactatgaattcagagtgtgtacactacttacatggttccacaccacccaccccagacacacacacacacacacacacaaacacacacacacaccctgcatccccacaaaaacacgacctgggcatcccaaattaagaatgctcattctgttattttgcttgtaatggttttcctatgaggcagatttttcaaaaacgataatgtccaccccaagtcgttataggtggaaaacatctcgcttggtatgaaataatctcccattcagtatgctttgggccccagacatagtgggagaggctggagtggaggcggggtggtttctagtctgggtgcctcttgtgggaggcactgggctgactgggtcaaccctccaggccctggcttaccggcgcccggccgtttctgctcgtaggtgctgtgtatgggagccatgggcgccgaggccgccagacctacacgcgctaccagacactggagctggagaaggagttccacttcaaccgctacctgacacggcgccgccgcatcgagatcgccaacgcgctctgcctcaccgagcgccagatcaagatctggttccagaaccgccgcatgaagtggaaaaaggaaaacaagctcatcaattccacgcagcccagcggggaggactcagaggcaaaggcgggcgagtagatgcctgggcagggaccaggccagcgctgcaacctccttcggctttgcccccttgccctcgcctgttccccaacttttctccccgcctgctcccatctgggggcttccgcagcttcaggggagcccggagctttgcaagcgtctgtgcatttatttcttacaaaacaaaacaaaacaaaacaaaacaaaacaaaacctcacacacagccaataccagcggtggagcggggcgcactgcacacacccagtcccgctccacaaggatgcccaaaccggctccggagttctggggtgcgccctgaatgcgtctggggctcctgtgctagtgtctctgagctccccgacctcgggacgcggggctgctggggaggaaagagccctgggccgagccccgtttcgtgtctgggcgtctctgcctaggtccctcgccagtaagtcccgaagggcatcatcaaatcatctctggccagtggaaagagggggtctaagagctggagcccggccttgtcgcggcctcccaagccgcctcccagaacaggaaggaagctcgaggaacaaagggggccccaacagagcccagtctctcggtcccgcgtgtgcaaccgtcagtggaagagaagcagcctcagccgaggcgagttaacctgggcgcgggtggaacattacagcccggggtaggctcccggctccggattcccgctcccagcctccgatagcgcccgcgtcgccgccacagcagcgttcaggacccaacgaggggcccagggccatgggagccgccagagtcctggcttccagacgtgccaggggcgcctgcaacgccgggcctccagcggggagactccacttgcctctcagctatatttgtgatttacttgagtctttcggagccgtggaaaataactacagagatactaggcactgccaaggaatgcttccattatcgccctcattttactcctggaggtctaagcaagaccccagtaacccgccccacggatccccagaccccaggcagcctctgtggctgcagggagcctgggcccactcgctgggttcaaggagaaccctccgacttggggcccggctggctcgaggaaccggaccctgtgatgaagattttccaggctggatacgtaagcaaatcaaatcgcaaactaatgacacgaaaaccatattcacacgaaagaaaaatcgactacggttataaaagtgtatggaatttgacctcgccttggagaaacactacacaaaagctatctttaatagacgcctgtcatttaagagcggcagggacactgtccctcatgcgctcgcaaaaacagagccgtaattgtagctgctgctgctgcgggcaggatttatttctccaattggctaaatggctttcccccttccccgaaggtgatatctgtattttcaaaattcagagctgctggcaggacggtcagtaccgaccccaacctcgacacaaaaataagaggggctgcaaaacgggggaaataaagttgttgtaaataaaatgcaagtcaccacctccccccaatcctactgcatcctcgccgggcgcgcgatcggcagctgacggcataacaattggtacatcctaatggaactgcgagggaaatgcaataattttgccataatgggctgtaacctcaattcgaccccggcccttgcagcccccggtcggaagctgggcgatgagccctgcctccagcgggtggcgctcgagtccggctgaacggcggcaactggcggcgggcacgcgcccggggcgcgcgcgccacccccctcgcctccacccaactcccctattagtgcacgagtttacctctagaggtcatcaggcaggatttacgactggacaacaaaagcacgtgattcgaagtcgtaccccatatttgggtgcctacgtaggagggaaccaagtacatgtcccagtcatttccataattcatcataaattgtgcaagggtgctatagacgcacaaacgacc

SEQ ID NO: 3 - protein, 249 aa, Homo sapiens
MGEHNLLNPGFVGPLVNIHTGDTFYFPNFRASGAQLPGLPSLSYPRRDNVCSLSWPSAEPCNGYPQPYLGSPVSLNPPFGRTCELARVEDGKGYYREPCAEGGGGGLKREERGRDPGAGPGAALLPLEPSGPPALGFNSSLLNEGNKGAGAGDPGSLVSPLNPGGGLSASGAPWYPINSRSRKKRKPYSKLQLAELEGEFLVNEFITRQRRRELSDRLNLSDQQVKIWFQNRRMKKKRLLLREQALSFF

SEQ ID NO: 4 - protein, 233 aa, Homo sapiens
MSSYFVNPTFPGSLPSGQDSFLGQLPLYQAGYDALRPFPASYGASSLPDKTYTSPCFYQQSNSVLACNRASYEYGASCFYSDKDLSGASPSGSGKQRGPGDYLHFSPEQQYKPDSSSGQGKALHDEGADRKYTSPVYPWMQRMNSCAGAVYGSHGRRGRQTYTRYQTLELEKEFHFNRYLTRRRRIEIANALCLTERQIKIWFQNRRMKWKKENKLINSTQPSGEDSEAKAGE

SEQ ID NO: 5 - DNA, 750 bp, Homo sapiens
atgggcgagcataatctcctgaatcccgggtttgtggggccgctggtaaacatccacacgggagacaccttctacttccccaacttccgcgcgtccggggcgcagcttcccgggctgccttcgctgtcctacccacgccgcgacaacgtgtgctccctgtcctggccgtcggcggagccgtgcaatggctacccgcagccctacctcggcagcccagtgtctctcaaccctcccttcggccgcacgtgcgagctggcgcgcgtggaggacggcaagggttactaccgcgagccgtgcgccgagggtggcggcgggggcctgaagcgtgaggagcgcgggcgcgacccgggagccgggcccggggcagcgctgctcccgctggagccgtcggggccgcctgcgctcggcttcaattcgtccctgctcaacgagggcaacaagggcgccggcgcaggcgaccccggcagcttggtatcgccgttgaaccccggcggcgggctctcggccagcggcgcgccctggtacccgatcaacagccgctctcggaagaagcgcaagccctattcgaagttgcaactggcagagctggagggcgagtttctggtcaacgagttcatcacacgccagcgccggagggaactctcagaccgcttgaatcttagtgaccagcaggtcaagatctggtttcagaaccggagaatgaaaaagaaaagacttctgttgagggagcaagctctctccttcttttaa

SEQ ID NO: 6 - DNA, 702 bp, Homo sapiens
atgagttcctattttgtgaatcccactttccccgggagccttcccagcggccaggactccttcttgggccagctgcccctctaccaggctggctatgacgcgctgaggcccttcccggcctcgtacggggcgtcgagtctcccggacaagacgtacacctcaccttgtttctaccaacagtccaactcggtcctggcctgcaaccgggcgtcctacgagtacggggcctcgtgtttctattctgataaggacctcagtggcgcctcgccctcgggcagtggcaagcagaggggccccggggactacctgcacttttctcccgagcagcagtacaaacccgacagcagcagcgggcagggcaaagcactccatgacgaaggcgccgaccggaagtacacgagcccggtttacccttggatgcagcggatgaactcctgcgcgggtgctgtgtatgggagccatgggcgccgaggccgccagacctacacgcgctaccagacactggagctggagaaggagttccacttcaaccgctacctgacacggcgccgccgcatcgagatcgccaacgcgctctgcctcaccgagcgccagatcaagatctggttccagaaccgccgcatgaagtggaaaaaggaaaacaagctcatcaattccacgcagcccagcggggaggactcagaggcaaaggcgggcgagtag